(19) United States
(12) Patent Application Publication
(10) Pub. No.: US 2008/0318884 A1
(43) Pub. Date: Dec. 25, 2008

(54) Methods for Altering Gene Expression and Methods of Treatment Utilizing Same

(75) Inventors: Peter J. Detloff, Birmingham, AL (US); Andrew B. Crouse, Madison, AL (US)

Correspondence Address: BRADLEY ARANT ROSE & WHITE, LLP;INTELLECTUAL PROPERTY DEPARTMENT-NWJ, 1819 FIFTH AVENUE NORTH, BIRMINGHAM, AL, 35203-2104, US

(73) Assignee: THE UAB RESEARCH FOUNDATION, Birmingham, AL (US)

(21) Appl. No.: 11/909,691

(22) PCT Filed: Mar. 27, 2006
(86) PCT No.: PCT/US06/11154

§ 371 Date: Sep. 25, 2007

Related U.S. Application Data
(60) Provisional application No. 60/665,203, filed on Mar. 25, 2005.

(51) Int. Cl. A61K 31/7105; C07H 21/02; A61P 21/00

(52) U.S. Cl. 514/44; 536/23.1

(57) ABSTRACT

The present disclosure describes methods for altering the expression of a target gene comprising a rare cluster of codons, including, but not limited to, trinucleotide repeats. The method utilizes, in part, on amino acid deprivation or the limiting of specific charged tRNAs. The methods for altering target gene expression may be used in treatment methods to treat diseases in a subject organism in need of such treatment. Such methods for altering target gene expression have not been heretofore recognized in the art. Exemplary diseases that may be treated using the methods of the present disclosure include any disease where altering the expression of the target gene would provide treatment. Such diseases include all forms of cancer, ageing, infectious disease, metabolic disorders, inflammation, neurological disorders, diabetes, psychiatric disorders and diseases associated with trinucleotide repeats.

### CROSS REFERENCE TO RELATED APPLICATIONS

This application claims priority to and the benefit of U.S. Provisional patent application No. 60/665,203, filed Mar. 25, 2005.

### FIELD OF THE DISCLOSURE

The present disclosure relates to methods for altering gene expression. The methods disclosed may be used in the treatment and/or prevention of a number of diseases, such as but not limited to, trinucleotide repeat diseases.

### BACKGROUND

Many diseases are caused, at least in part, through inappropriate expression of one or more genes. For the present disclosure, such genes may be endogenous to the subject organism or may be genes that are expressed from an infectious organism (such as but not limited to, a virus, a bacteria, and/or a parasite). For example, tumor formation and progression depends upon the altered expression of one or more genes. Infectious agents are also dependent on the expression of genes including, but not limited to, antibiotic resistance genes. Additionally, in inherited diseases a gene may contain a mutation that contributes to the initiation and/or progression of a disease. The mutation may be present on one or both of the alleles of the gene and may or may not impact the level or amount of the polypeptide encoded by the gene or the function of the polypeptide encoded by the gene. The mutation may also cause its effect without being translated by mechanisms solely dependent on alteration of the sequence of a messenger RNA. The mutation may be a silent mutation. The type of mutation present in the gene can suggest options for the treatment of the disease associated with the mutation.

One example of inherited disorders is the trinucleotide repeat disorders. The sequence of every messenger RNA that codes for a protein contains combinations of nucleotides, consisting of 3 nucleotides each, called codons. The sequence of a codon directs the ribosome to use a particular tRNA to add a particular amino acid during the translation of mRNA to protein. In some cases, codons are repeated in close proximity many times within a single mRNA. Since a codon length is three nucleotides, such repeats are termed “trinucleotide repeats”. Certain trinucleotide repeats, when expanded above a certain length are known to cause a disorder/disease. As a group these are referred to as trinucleotide repeat diseases. When the trinucleotide repeat expansion reaches a certain length, referred to as the critical length, the disease process is initiated. The critical length varies for each given disease. A number of repeat diseases have recently been identified and include, but are not limited to Huntington's disease (HD), spinobulbar muscular atrophy (SBMA), dentatorubral-pallidoluysian atrophy (DRPLA), spinocerebellar ataxia types 1, 2, 3, 6, 7 and 17 (SCAs) (the foregoing each caused by a CAG trinucleotide repeat), oculopharyngeal muscular dystrophy (OPMD), congenital hypoventilation syndrome (CCHS), holoprosencephaly, infantile spasm syndrome, mental retardation, X-linked, with isolated growth hormone deficiency, cleidocranial dysplasia (CCD), synpolydactyl), hand-foot-genital syndrome, and blepharophimosis/ptosis/epicanthus inversus syndrome (BPEIS), (the foregoing each caused by a GCG trinucleotide repeat). Additionally, pseudoachondroplasia/MED is caused by either an expansion or contraction of a GAC repeat (1). As a result of the trinucleotide repeat, the polypeptides encoded by these genes may contain an expanded repeat of the amino acid coded for by the triplet expansion. For example, the CAG triplet expansion in HD, SBMA, DRPLA and the SCAs codes for an expanded glutamine repeat, the GCG triplet expansion in OPMD, CCHS, holoprosencephaly, infantile spasm syndrome, mental retardation, CCD, synpolydactyl), hand-foot-genital syndrome, and BPEIS codes for an expanded alanine repeat and the GAC repeat in pseudoachondroplasia/MED codes for an expanded aspartate repeat.

In many cases, the trinucleotide repeat diseases are dominant, meaning that the inheritance of only one copy of a gene containing the triplet expansion is sufficient to cause the disease. The trinucleotide repeat expansion may cause a toxic gain of function not related to the normal function of the gene. Strong evidence exists for a gain-of-function mechanism for the expanded CAG repeat that causes SBMA and HD, since other mutations that cause a loss of function in these genes do not result in the disease phenotype. These diseases generally exhibit autosomal dominant inheritance and almost all of the afflicted patients express a normal allele in addition to the long repeat allele. Therefore, many diseases may be treated by selectively inhibiting the expression of the mutant allele of the gene while leaving the expression of the wild type allele unaffected. Several lines of evidence support the view that reducing the expression of the mutant gene coding for the expanded trinucleotide repeat will provide therapeutic benefit. However, strategies to reduce the expression of the mutant gene coding for the expanded amino acid repeat often have the unintended consequence of causing potentially lethal side effects arising from or related to loss of gene function from both alleles.

There are several reduction of expression strategies that are sequence based that could be used to specifically decrease expression of a gene or a single allele of a gene. These include, but are not limited to, antisense RNA, ribozyme, DNA enzyme and RNA interference based methods (2-5). These methods share an important property with the method described in the current disclosure in that they are designed to destroy mRNA needed to make the altered polypeptide. Since a single mRNA can be used to produce many (even thousands) of altered polypeptides this is a great advantage over methods designed to solely decrease protein amounts. Another advantage of targeting mRNA is in the case of untranslated trinucleotide repeat disorders, such as myotonic dystrophy 1 and 2, in which the mRNA, as opposed to the protein, is thought to be the molecule that contributes to the initiation or progression of the disease. RNA interference (RNAi) is perhaps the most promising of these methods and several advancements in our understanding of the machinery involved in RNAi have brought this method closer to providing a therapy (6). RNAi has not been effective directly against long CAG repeats, the defining difference between disease and wild type alleles (3, 7). This might be due to an unusual structure of the repeats in mRNA or simply that CAG is not a sequence that is recognized by the RNAi machinery of mammalian cells (most sequences are not). A possible explanation for the failure of such sequence based methods to act on specific desirable target sequences is that these target sequences of an mRNA may be inaccessible due to the presence of ribosomes translating the mRNA. This possibility is related to the present disclosure, since the described method may make previously unavailable sequences available for targeted destruction by one or more of the aforementioned methods. Presently the art concerning allele specific degradation of repeat coding mRNAs relies on an indirect method exemplified by the work of Miller et al. who have recently devised a means for allele specific RNA interference therapy. By using small interfering RNAs (siRNAs) targeting allelic differences other than the CAG repeat in the MJD (SCA type 3) disease gene they have shown an allele specific reduction of CAG repeat containing mRNAs in mammalian cells (7). This method has several drawbacks and does not yet provide means of treatment. First, since most sequences are not amenable to RNAi, it is not clear how many transcribed polymorphisms will be useful for allele specific reduction of expression. Such sequences must be present as heterozygosities in many patients to be generally useful. The successful application of this approach will require customization for each patient. Take for example HD, where transcribed sequence variants are found on both the expanded and wild type alleles (8). In some individuals a variation will be present on the expanded CAG transcript and in others this same variation would be present on the normal length CAG transcript. Thus, targeting a heterozygosity would require the determination of which transcript variant contains the expanded allele for each patient—a difficult and time consuming task given the great length of the HD mRNA. Another major obstacle for effective therapy by these sequence-based methods is safe and efficient delivery of oligonucleotide. These challenges suggest this indirect strategy will require many more years of study and then apply only to those lucky enough to have the right combination of linked transcript variants.

The present disclosure describes methods for altering the expression of a gene encoded by mRNAs comprising a rare cluster of codons. The methods are based on altering the availability of specific amino acids or their cognate aminoacylated tRNAs needed for translation of such rare clusters of codons within the target mRNA. The methods for reducing gene expression may be used in treatment methods to treat diseases in a subject organism in need of such treatment. Such methods for reducing gene expression have not been heretofore recognized in the art. Exemplary diseases that may be treated using the methods of the present disclosure include trinucleotide repeat diseases, including but not limited to, Huntington's disease, spinobulbar muscular atrophy, dentatorubral-pallidoluysian spinobulbar muscular atrophy, spinocerebellar ataxia types 1, 2, 3, 6, 7 and 17, oculopharyngeal muscular dystrophy, congenital hypoventilation syndrome, holoprosencephaly, infantile spasm syndrome, mental retardation, X-linked, with isolated growth hormone deficiency, cleidocranial dysplasia, synpolydactyl), hand-foot-genital syndrome, and blepharophimosis/ptosis/epicanthus inversus syndrome, pseudoachondroplasia/MED (1). In addition, exemplary diseases that may be treated using the methods of the present disclosure include those diseases which require or are influenced by the expression of a gene comprising a rare cluster of codons. Alternately, this method could be used to alter gene expression levels of genes that contain a rare cluster of codons and contain disease causing mutations in untranslated regions of the gene (e.g. the expanded CTG repeat associated with Myotonic Dystrophy). Furthermore, the present disclosure provides for methods to increase the expression of target genes by methods that alter the levels of specific amino acids or their cognate tRNAs that may be present in a rare cluster of codons Furthermore, this method can be used to alter the expression levels of genes that may or may not contain mutations.

### BRIEF DESCRIPTION OF THE FIGURES

FIG. 1 illustrates the onset of abnormalities in HdhQ150 heterozygote and homozygote mice in tail suspension, gait and cage activity trials.

FIGS. 2A-C show that glutamine deprivation and inhibition of tRNA charging decreases gene expression. FIG. 2A shows the effect of glutamine deprivation on the steady state mRNA levels of wild type Hprt and HprtQ150. FIG. 2B shows the effect of glutamine deprivation on the steady state mRNA levels of wild type Hprt and HdhQ150. FIG. 2C shows the effect of inhibition of tRNA charging via the glutaminyl-tRNA synthetase inhibitor QSI on the steady state levels of wild type Hdh and HdhQ150.

FIG. 3 illustrates a potential mechanism to explain reduction of long amino acid repeat mRNA levels by amino acid deprivation.

FIGS. 4A-C show a mathematical model to explain the effect of reduced amino acid availability on gene expression.

FIGS. 5A and B show the effect of amino acid deprivation on mRNA stability of HdhQ150 and HprtQ150 mRNAs.

FIG. 6 shows a potential means of combining amino acid deprivation therapy with a sequence based reduction of expression therapies.

FIG. 7 shows two potential mechanisms for implementing glutamine deprivation therapy.

### DETAILED DESCRIPTION

The present disclosure describes methods for altering the expression of a target gene coding for mRNAs comprising a rare cluster of codons. The methods are based on altering the availability of specific amino acids or their cognate aminoacylated tRNAs needed for translation of such rare clusters of codon within the target mRNA. In one embodiment, the levels of the cognate tRNAs are reduced and target gene expression is decreased; in an alternate embodiment, the levels of cognate tRNAs are increased and target gene expression is increased.

As discussed above, at least ten late-onset neurological diseases are caused by the inheritance of a gene coding for a protein with an expanded glutamine (Gln) repeat. These diseases, include, but are not limited to, Huntington's disease (HD), spinobulbar muscular atrophy (SBMA), dentatorubral-pallidoluysian atrophy (DRPLA), and several of the Spinocerebellar ataxia types 1, 2, 3, 6, 7 and 17 (SCAs) (18,19). In addition, at least nine diseases are caused by the inheritance of a gene coding for a protein comprising an expanded alanine (Ala) repeat. These diseases, include, but are not limited to, oculopharyngeal muscular dystrophy (OPMD), congenital hypoventilation syndrome (CCHS), holoprosencephaly, infantile spasm syndrome, mental retardation, X-linked, with isolated growth hormone deficiency, cleidocranial dysplasia (CCD), synpolydactyl), hand-foot-genital syndrome, and blepharophimosis/ptosis/epicanthus inversus syndrome (BPEIS). Furthermore, at least one disorder is caused by the inheritance of a gene coding for an aspartate (Asp) repeat, namely pseudoachondroplasia/MED (1).

As an exemplary disease, HD is discussed in detail to illustrate the teachings of the present disclosure. The use of HD as an exemplary disease is not meant to limit the application of the teachings of the present disclosure to HD. As discussed above, the teachings of the present disclosure can be applied to reduce the expression of any target gene comprising a rare cluster of codons.

HD is an autosomal dominant neurological disorder involving involuntary movements, psychiatric disturbances and cognitive impairment (20). Symptoms typically present during mid-life and progress until death 15 to 20 years after onset. Postmortem analysis reveals degeneration in several areas of the brain with prominent cell loss in the striatum (21). HD is caused by the inheritance of a CAG repeat greater than 35 units in length in exon 1 of a gene of unknown function called huntingtin (22, 23). Longer repeats are associated with earlier ages of onset and short repeats (less than 36 CAGs) are found in individuals not affected by HD (24). The trinucleotide repeat codes for a polyglutamine stretch near the N-terminus of the huntingtin protein, and the polyglutamine contributes to protein aggregates found in affected regions of patient brains (25, 26). The molecular steps mediating neurotoxicity in HD and the other CAG/polyglutamine diseases remain unknown. The nucleotide sequence for the human HD gene and its corresponding amino acid sequences are shown in SEQ ID NOS. 1 and 2. The nucleotide sequence for the murine homologue, the Hdh gene, and its corresponding amino acid sequences are shown in SEQ ID NOS. 3 and 4

The translated CAG trinucleotide repeat diseases may share a common molecular mechanism in their etiology. This hypothesis is supported by the applicant's previous work in which they showed mice containing an insertion of a long (150) CAG repeat into a gene unrelated to the CAG/polyglutamine repeat disorder genes (the mouse Hprt locus) share a similar presentation of symptoms to the CAG repeat disorders of man (27).

For each of the trinucleotide repeat diseases the molecular mechanism of pathogenesis may involve multiple pathways. For HD this view is supported by the existence of many interacting partners of the wild type huntingtin protein and the potential to partner with many other proteins by polyglutamine-polyglutamine interactions (28-33). The processes that have been reported to be affected by mutant huntingtin range from mitochondrial function (34) to transcription (35) to glutamate uptake by vesicles in neurons (36) to vesicular transport (37). Additionally, for each of the trinucleotide repeat diseases a direct role for the disease mRNA has not been ruled out. The possibility that the CAG repeat in mRNA directly causes some or all of the toxicities of these diseases has an established precedent. The trinucleotide repeat that causes Myotonic Dystrophy is an untranslated CTG repeat that needs only be transcribed to mRNA to inhibit a vital cellular function (38). Furthermore, there exist specific proteins that bind CAG repeats in mRNA and this interaction has been implicated in the pathology of CAG repeat disorders (39). By this view reduction of disease protein would not be therapeutically beneficial unless the mRNA levels were also reduced. This highlights one of the advantages of methods that reduce mRNA levels such as the one disclosed in this application.

The diversity of cellular mechanisms affected by trinucleotide repeat mutations may make these diseases difficult to treat by rational drug design specifically targeting each of these affected processes. The problem of molecular diversity underscores the advantage of potential therapies designed to reduce HD gene expression, in particular when the disease mRNA level is reduced, since such strategies might reverse the effects of the mutation on all affected processes. This approach is further supported by evidence that many of the trinucleotide repeat mutations cause pathology by gain-of-function mechanisms.

Using the present example of HD, the benefits of reducing huntingtin expression have been shown in animal models by the comparison of transgenic mice with varying levels of expression. Higher levels of HD transgene expression correlate with an earlier onset of HD-like symptoms in mice (40, 41). Furthermore, eliminating expression of an HD transgene with and expanded CAG/polyglutamine repeat reverses an HD-like pathology in mice (42). Even slight reductions in huntingtin expression may reduce or prevent the clinical manifestation of HD, since HD can take decades before onset and still decades more during its progressive course.

As discussed above, strategies to reduce gene expression have considerable disadvantages. For example, Hdh (the mouse homolog of the HD gene) is necessary in development and knockout mice exhibit lethality early in embryogenesis (43-45). Reducing huntingtin protein expression to less than 30% of wild type levels impairs neurogenesis (46), and removal of the Hdh gene from the forebrain late in development (5 days post partum) causes progressive neurodegeneration (47). Chimera analysis showed murine embryonic stem (ES) cells lacking Hdh gene expression did not contribute to some brain regions. Furthermore, these chimeras showed a number of abnormalities (48). These results suggest that a downregulation strategy, if carried too far, might cause harm to some brain regions. Nevertheless, loss of only one Hdh allele, which is known to decrease brain expression to approximately half of normal, is compatible with normal development and neurological function in mice (44, 45). Furthermore, humans with balanced translocations through the HD gene lack abnormalities (49).

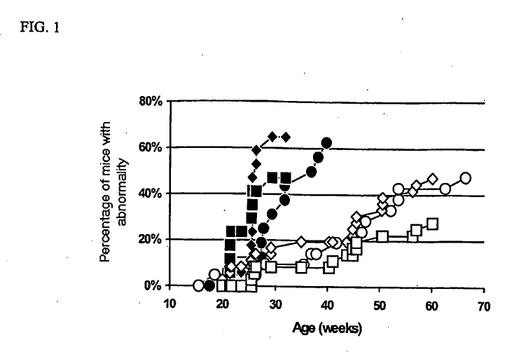

The onset and severity of HD-like symptoms has been correlated with the expression levels of the mutant Hdh gene. For example, earlier onset of HD-like abnormalities are observed in Hdh^{(CAG)150} homozygote mice than in heterozygote mice suggesting that levels of gene expression have an effect on the severity of the disease in mice (FIG. 1). In FIG. 1, filled symbols indicate Hdh^{Q150} homozygotes and open symbols indicate Hdh^{Q150} heterozygotes. Diamonds represent mice in a tail suspension trial. Mice were classified as abnormal if the mice clasped in one or more of ten trials (n=18 homozygotes, n=35 heterozygotes). Circles represent mice in the gait analysis trials (n=16 homozygotes, n=21 heterozygotes). A gait overlap mean greater than 0.8 cm was considered abnormal. Squares represent mice in the open cage activity trial (n=18 homozygotes, n=35 heterozygotes). As can be seen in FIG. 1, mice homozygous for HdhQ150 displayed earlier onset of abnormalities than heterozygotes in each of the trials.

These findings suggest that strategies to reduce the expression of the HD mutant allele while maintaining the expression of the wild type allele might provide therapeutic benefit while leaving the subject with enough normal (or wild-type) gene product to maintain proper cellular function. Alternatively, decreasing expression from each allele could also provide benefit.

Selective alteration, or allele specific alteration of gene expression, is possible using the teachings of the present disclosure. Using murine embryonic stem (ES) cells expressing knock-in versions of the mouse Hdh gene (Huntington's disease homolog) comprising an expanded CAG repeat of 150 glutamine codons and the mouse Hprt gene comprising an expanded CAG repeat of 150 glutamine codons, the effect of amino acid deprivation on gene expression was examined. The present disclosure shows that glutamine deprivation reduces the levels of mRNA coding for long glutamine repeat alleles without reducing mRNA from short glutamine repeat alleles. The underlying mechanism involves selective destabilization of mRNA expressed from the expanded trinucleotide repeat allele. Therefore, agents designed to induce glutamine deprivation or mimic the molecular effects of glutamine deprivation (such as but not limited to decreased levels of aminoacylated glutaminyl-tRNA) should selectively reduce the levels of the polyglutamine coding mRNAs related to disease.

Therefore, it is an object of the present disclosure to provide a method to alter the gene expression of a target gene, said target gene encoded by an mRNA comprising a rare cluster of codons. In a specific embodiment, when such a rare cluster of codons contains a polymorphic sequence variation the method described by the present disclosure allows allele specific alteration of mutant gene expression.

In a specific embodiment, target gene expression is reduced by altering the availability of one or more amino acids coded for by the rare cluster of codons within said target gene. The reduction in availability of the one or more amino acids may be a total reduction or a partial reduction. A variety of mechanisms may be used to reduce the availability of the one or more amino acids. In one embodiment, reduction in availability occurs by reducing the dietary intake of the amino acid. In an alternate embodiment, the reduction in availability occurs by generally inhibiting or reducing the endogenous synthesis of the amino acid. In another alternate embodiment, the transport of the amino acid to a particular target tissue is inhibited. In another alternate embodiment, substrates for a chemical reaction that consumes the amino acid are provided. In another alternate embodiment, treatment that stimulates the sequestration of the amino acid from one tissue or compartment at the expense of another are applied. In yet another alternate embodiment physical methods, such as but not limited to dialysis, are used to remove the desired amino acid. In still a further embodiment, one or more of the preceding methods are used in combination with one another. In another alternate embodiment, the aminoacylated tRNA for the amino acid is eliminated or reduced.

In a specific embodiment, target gene expression is increased by increasing the availability of one or more amino acids coded for by the rare cluster of codons within said target gene. A variety of mechanisms may be used to increase the availability of the one or more amino acids. In one embodiment, an increase in availability occurs by increasing the dietary intake of the amino acid. In an alternate embodiment, the increase in availability occurs by generally stimulating or enhancing the endogenous synthesis of the amino acid. In another alternate embodiment, the transport of the amino acid to a particular target tissue is increased. In another alternate embodiment, other chemical reactions that consume the amino acid are inhibited. In another alternate embodiment, treatment that stimulates the sequestration of the amino acid from one tissue or compartment is applied. In yet another alternate embodiment physical methods, perenteral nutrition, are used to directly add the amino acid to the blood stream. In another alternate embodiment, of the level aminoacylated tRNA for the amino acid is increased by pharmacological or genetic means. In a specific embodiment, target gene expression is increased by starving the amino acids of a rare cluster of codons downstream of an endogenous pause site. In still a further embodiment, one or more of the preceding methods are used in combination with one another.

Mutations are not required for altering target gene expression by the methods described in this disclosure. Nevertheless, the targeting of a mutation by this method provides a means of altering the expression of the mutant allele selectively.

It is an additional object of the disclosure to use such methods to reduce gene expression of a target gene comprising a rare codon cluster to provide a treatment method to treat and/or prevent a disease state in a subject organism in need of such treatment. The disease may be a trinucleotide repeat disease or a disease requiring the expression of a gene comprising a rare cluster of codons. The subject organism may be any animal, virus, bacteria or plant that utilizes nucleic acid to direct the production of a polypeptide. In a specific embodiment, the subject organism is a mammal, such as a human. The treatment method need not absolutely reduce the expression of the target gene. A reduction in gene expression will have beneficial effects to the treatment and/or prevention of the disease state. In one embodiment, the treatment method prevents or reduces the clinical manifestation of said disease state. In an alternate embodiment, the treatment method delays the onset of the clinical manifestations of the disease state.

The treatment method may reduce the expression of one or both alleles of the target gene. In one embodiment, the target gene is heterozygous with the two alleles differing in the occurrence of codons that specify the amino acid or amino acids whose availability is reduced (i.e., the rare codon cluster).

In a specific embodiment, the disease state comprises a polyglutamine disorder. Examples of polyglutamine disorders, include, but are not limited to, Huntington's disease (HD), spinobulbar muscular atrophy (SBMA), dentatorubral-pallidoluysian atrophy (DRPLA), and spinocerebellar ataxia types 1, 2, 3, 6, 7 and 17 (SCAs). In another specific embodiment, the disease state comprises a polyalanine disorder. Examples of polyalanine disorders, include, but are not limited to, oculopharyngeal muscular dystrophy (OPMD), congenital hypoventilation syndrome (CCHS), holoprosencephaly, infantile spasm syndrome, mental retardation, X-linked, with isolated growth hormone deficiency, cleidocranial dysplasia (CCD), synpolydactyl), hand-foot-genital syndrome, and blepharophimosis/ptosis/epicanthus inversus syndrome (BPEIS). In another specific embodiment, the disease state comprises a polyaspartate disorder. An example of a polyaspartate disorder is Pseudoachondroplasia/MED which is caused by either an expansion or contraction of a GAC repeat (1).

It is another object of the disclosure to provide such treatment methods to alter the expression of a target gene in combination with a second means of altering gene expression to provide an additive, synergistic or more selective alteration in expression of said target gene. The second means for altering gene expression may act in an allele specific manner or an allele non-specific manner.

### DEFINITIONS

The terms “prevention”, “prevent”, “preventing”, “suppression”, “suppress” and “suppressing” as used herein refer to a course of action initiated prior to the onset of a clinical symptom of a disease state so as to prevent or reduce a clinical manifestation of the disease state. Such preventing and suppressing need not be absolute to be useful.

The terms “treatment”, “treat” and “treating” as used herein refers a course of action initiated after the onset of a clinical symptom of a disease state so as to eliminate or reduce a clinical manifestation of the disease state. Such treating need not be absolute to be useful.

The term “in need of treatment” as used herein refers to a judgment made by a caregiver that a patient requires or will benefit from treatment. This judgment is made based on a variety of factors that are in the realm of a caregiver's expertise, but that includes the knowledge that the patient is ill, or will be ill, as the result of a condition that is treatable by a method or compound of the disclosure.

The term “in need of prevention” as used herein refers to a judgment made by a caregiver that a patient requires or will benefit from prevention. This judgment is made based on a variety of factors that are in the realm of a caregiver's expertise, but that includes the knowledge that the patient will be ill or may become ill, as the result of a condition that is preventable by a method or compound of the disclosure.

The term “individual”, “subject”, “subject organism”, “host organism” or “patient” as used herein refers to any animal or plant, including mammals, such as mice, rats, other rodents, rabbits, dogs, cats, swine, cattle, sheep, horses, or primates, and humans. The term may specify male or female or both, or exclude male or female.

The term “rare codon”, “rarely used codon” or “underrepresented codon” as used herein refers to a codon whose use is underrepresented when compared to all other codons in all known open reading frames in a host organisms; in one embodiment, a “rare codon”, “rarely used codon” or “underrepresented codon” refers to a codon that is used on average in a host organism less than 17 times per 1000 codons; in an alternate embodiment, a “rare codon”, “rarely used codon” or “underrepresented codon” refers to a codon that is used on average in a host organism less than 8 times per 1000 codons.

The term “rare cluster of codons”, as used herein refers to one or more codons within the coding sequence of the mRNA of a target gene, such that the one or more codons is present in few or no other genes in the host organism. A rare cluster of codons can be as few as three codons; there is no upper limit on the size of a rare cluster of codons. In one embodiment, the rare cluster of codons comprises a sequence of the same codon (such as a trinucleotide repeat); the sequence comprising the same codon may be a contiguous sequence (meaning no other codons are dispersed within the sequence) or the sequence comprising the same codon may be a non-contiguous sequence (meaning that other codons are present within the sequence, provided that the repeated codon comprises at least 50% of the codons within the non-contiguous sequence). In an alternate embodiment, the rare cluster of codons may comprise one or more underrepresented codons; the sequence comprising the one or more underrepresented codon may be contiguous (meaning no other codons are dispersed within the sequence) or the sequence comprising the underrepresented codon may be non-contiguous (meaning that other codons are present within the sequence, provided that the underrepresented codons comprises at least 50% of the codons within the sequence). In yet another alternate embodiment, the rare cluster of codons comprises a unique sequence of codons (which may be underrepresented codons or codons that are not underrepresented) that is not present in any other mRNA, or that is present in a few other mRNAs (as used in this specification, “a few other mRNAs” shall mean less than 0.5% of the total mRNAs of a host subject, less than 1% of the total mRNAs of a host subject, less than 2.5% of the total mRNAs of a host subject or less than 5% of the total mRNAs of a host subject). Other examples of rare clusters of codons may be envisioned with the embodiments above provided for exemplary purposes only.

The term “target RNA” refers to any RNA molecule that contains a rare cluster of codons. The target RNA in one embodiment is an mRNA.

The term “target gene” as used herein refers to an gene whose expression in directed by a target RNA.

The term “therapeutically effective amount” as used herein refers to an amount of a molecule, either alone or as a part of a pharmaceutical composition, that is capable of having any detectable, positive effect on any symptom, aspect, or characteristics of a disease state. Such effect need not be absolute to be beneficial.

Gene Expression of Hprt mRNA Correlated with Amino Acid Concentration

In initial experiments, it was discovered that altering the reading frame of the CAG repeat in mouse Hprt mRNA drastically impacted the stability of the mRNA (the nucleotide sequence of the mouse Hprt gene is shown in SEQ ID NO. 5 and its corresponding amino acid sequence shown in SEQ ID NO. 6). In these experiments the reading frame was altered from glutamine to the other two possible reading frames for this repeat (alanine and serine). The mouse brain levels of Hprt mRNA with 150 CAGs coding for glutamine, alanine or serine were 50%, 23% and 2% of wild type levels, respectively. Since these were gene targeted alleles where the 150 CAGs trinucleotide repeat were inserted into the same position of the endogenous locus with the same transcriptional promoter, the transcription rate was most likely similar for each of the alleles. When mRNA stability was measured for each of the three reading frames in ES cells' where de novo transcription was inhibited, it was found that the serine frame mRNA had a half life of 3 hours whereas the glutamine and alanine frame mRNAs had half-lives greater than 10 hours. The decreased stability of the mRNA with the serine frame is consistent with the decreased brain HprtS150 mRNA levels.

The relative levels of Hprt Q150, A150 and S150 mRNAs in the brain roughly correlated with previously published bloodstream levels of the amino acids (glutamine>alanine>serine, (50)). This correlation suggested that the decreased levels of HprtS150 mRNA and the decreased stability of this mRNA were related to amino acid levels.

Altering The Levels of a Specific Amino Acid Selectively Alters the Expression of Alleles in Target Genes Comprising a Rare Cluster of Codons Comprising that Amino Acid

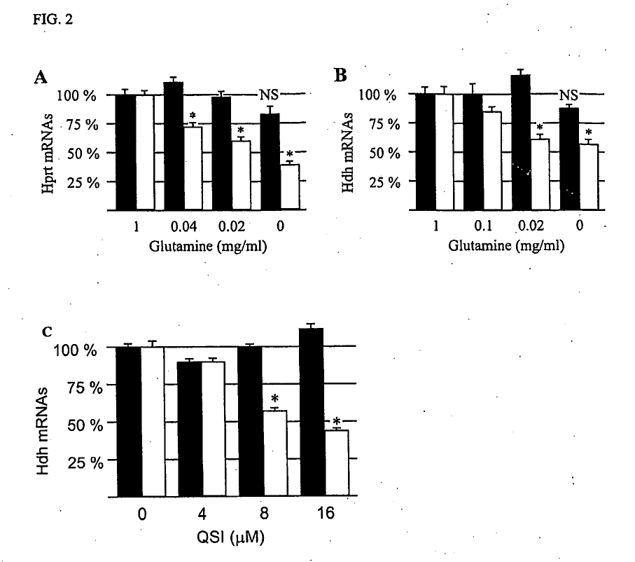

Knock-in murine embryonic stem cells hemizygous for alleles expressing repeats of 150 glutamines from either the mouse Hprt locus (hprtQ150) (the amino acid sequence is shown in SEQ ID NO. 8) or mouse Huntington's Disease homolog (HdhQ150) (the amino acid sequence is shown in SEQ ID NO.7) were obtained as described in the Methods section. These 150 codons for glutamine are examples of rare clusters of codons, specifically an expanded trinucleotide repeat. The ES cells were treated with methionine sulfoximine (MSO) to inhibit cellular glutamine synthesis while varying the levels of exogenous glutamine in the ES cell culture media. These steps reduce the availability of glutamine in the ES cells. Reduction of glutamine resulted in graded decreases of the steady state levels of mRNAs containing long CAG triplet expansions without significantly reducing the steady state levels of the wild type mRNAs (FIGS. 2A and B). FIG. 2A shows the relative levels of Hprt mRNA determined by quantitative real-time PCR in ES cells subject to varying glutamine concentrations. Filled bars represent wild type Hprt mRNA levels and open bars represent HprtQ150 mRNA levels. Error bars represent the SEM of 6 to 9 replicates. Asterisks indicate significant differences in comparison to wild type Hprt mRNA at the same glutamine concentration and a significant difference from the HprtQ150 allele mRNA level for 1 mg/ml glutamine (p<0.004 Mann-Whitney). “NS” indicates no statistical difference compared to wild type Hprt mRNA level from cells in 1 mg/ml glutamine. FIG. 2B shows the relative levels of Hdh mRNA determined by quantitative real-time-PCR in ES cells subject to varying glutamine concentrations. Filled bars represent wild type Hdh mRNA levels from a single allele and open bars represent mRNA levels from a single Hdh Q150 knock-in allele. Error bars represent the SEM of 6 to 9 replicates. Asterisks indicate significant differences in comparison to wild type at the same glutamine concentration and a significant difference from the HdhQ150 allele mRNA level for 1 mg/ml glutamine (p<0.004 Mann-Whitney). “NS” indicates no statistical difference compared to wild type Hdh mRNA level from cells in 1 mg/ml glutamine.

These results reveal that altering the amounts of glutamine available to the ES cells directly correlates with alterations in levels of HprtQ150 and HdhQ150 mRNAs while levels of wild type versions of these mRNAs remain normal regardless of glutamine concentration. Therefore, mRNA levels were correlated with the presence of a rare cluster of codons encoding the amino acid whose concentration was varied.

Inhibition of tRNA Aminoacylation Selectively Alters the Expression of Genes Comprising a Rare Cluster of Codons

ES cell lines hemizygous for HdhQ150 were treated with an inhibitor of the glutaminyl-tRNA synthetase, QSI (5′-O-[N-(L-glutaminyl)sulfamoyl]adenosine) for six hours in the presence of high levels of glutamine. HdhQ150 mRNA levels were lowered by QSI treatment in a dose dependent manner (FIG. 2C). Control ES cells expressing a single wild type Hdh allele showed that the effect of QSI in lowering Hdh mRNA levels was dependent on the presence of the long CAG repeat. In this case the long CAG repeat is used to represent a rare cluster of codons. This diminishment in disease allele expression was accomplished in the presence of high amounts of glutamine (1.2 grams/ml) showing that reduction of expression can be attained by inhibition of a tRNA synthetase while providing normal amounts of the amino acid which is a substrate for the synthetase reaction. Thus therapy might be attained by inhibition of tRNA synthetase reactions without the need for reduction of levels of cognate amino acids.

Filled bars represent wild type Hdh mRNA levels and open bars represent HdhQ150 mRNA levels. Error bars represent the SEM of 9 to 12 replicates. Asterisks indicate significant differences in comparison to wild type at the same QSI concentration and a significant difference from the HdhQ150 allele mRNA level in 0 μM QSI (p<0.0001 Mann-Whitney).

A number of mechanistic explanations could account for the decreased mRNA levels observed in FIGS. 2 A-C. While not being limited to a particular mechanism of actions, one possible explanation involves alterations in the translation of the long repeat mRNA. Translation is a process that is integrally linked to mRNA decay in prokaryotes and eukaryotes. Inhibition of translation initiation and elongation alter the stability of many eukaryotic mRNAs (reviewed in (51)). Translation initiation factors eIF4E and eIF4G are involved in stabilization of mRNA (52). Translation also alters the effects of cis-acting stability determinant regions that are found within open reading frames of several genes. Examples of mRNAs with such motifs include mammalian c-fos and c-myc and yeast MATalpha1 (9-13). Interestingly, the Hdh mRNA has a region of high homology with the translation sensitive c-myc CRD instability element (66% identity). An additional link between translation and mRNA stability is the influence of underrepresented codons. In yeast there is a direct correlation between the presence of underrepresented codons and the stability of mRNAs in general (14). Furthermore, experiments with MATalpha1 and c-myc show that underrepresented codons play a major role in the functioning of their instability determinants (10, 11). The implication of the underrepresented codon effect is that the cognate aminoacylated tRNA might be limiting, resulting in ribosome pausing at an underrepresented codon which in turn allows degradation of the mRNA via the CRD. Thus a potential mechanism of the glutamine deprivation involves pausing of the ribosome within a long CAG repeat which enhances the destruction of the mRNA. In other words, rare clusters of codons, such as expanded glutamine repeats, may mimic underrepresented codon effects on mRNA stability when an amino acid coded for by the rare cluster of codons is limiting.

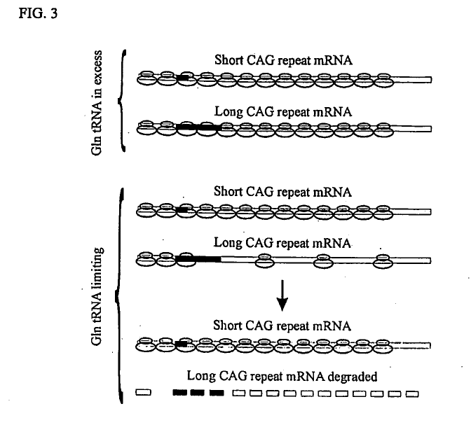

This mechanism is illustrated in FIG. 3. The open boxes represent mRNAs with the black boxes representing a rare cluster of codons (in this case a CAG trinucleotide repeat regions coding for glutamine). Each double oval represents a translating ribosome. Under normal conditions (upper panel) the rate limiting factor for translation elongation is not dependent on glutamine levels as glutamine and the charged glutamine-tRNA are both present in excess. As shown in the upper panel, transcription of the mRNA containing the CAG trinucleotide repeat region proceeds normally. As illustrated in the lower panel, decreasing glutamine levels generally or decreasing the supply of the charged glutamine-tRNA causes ribosome pausing during translation of longer CAG trinucleotide repeat regions, but not during the translation of short CAG trinucleotide repeat regions. The paused ribosome allows mRNA degradation of long repeat mRNAs.

The glutamine codon CAG is an abundant one in both mice and humans (53). Under normal conditions, levels of polypeptides with long CAG/polyglutamine repeats are approximately equivalent to wild type (containing short CAG/polyglutamine repeats) suggesting that the amount of charged glutamine-tRNA available for protein synthesis is not limiting the protein concentration (54, 55). Nevertheless, the levels of Hdh mRNA in the brains of mice and in ES cells were carefully measured and it was found that the insertion of a 150 CAG trinucleotide repeat (in the glutamine reading frame) causes a mild reduction in Hdh mRNA level (40 to 70% of wild-type (56)). Further reduction is caused by glutamine deprivation as discussed above. If each additional codon in a amino acid repeating sequence enhances the probability of destruction of an mRNA, then mRNAs with long trinucleotide repeats could be greatly affected by very small reductions in specific aminoacylated-tRNA levels. By such a model the iterative nature of the repeat would make long repeat messages less stable.

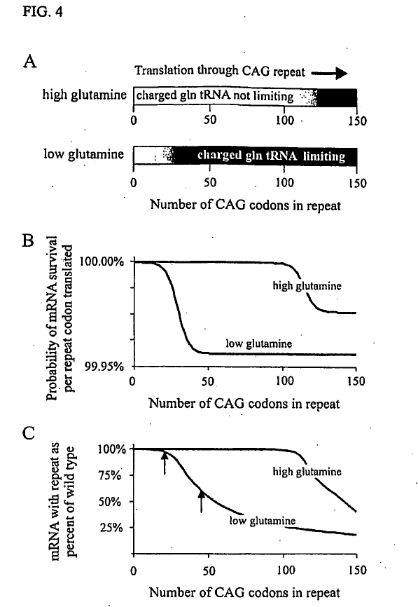

As an illustration of the leverage such iteration could have, a mathematical model is illustrated in FIGS. 4A-C. This model uses a glutamine repeat as an example of a rare cluster of codons, but would apply to any rare cluster of codons. Under normal conditions the amount of aminoacylated tRNAs available to a translating ribosome exceeds the rate of consumption making other aspects of translation rate limiting for elongation. The translation of a long CAG trinucleotide repeat starts with high levels of charged glutamine-tRNA which is rapidly depleted during translation of the repeat to a point where its transient local concentration becomes limiting. The resulting translational pauses would occur with greater likelihood in the downstream regions of a long trinucleotide repeat (represented by a darkening of the upper box in panels in FIG. 4A). Limiting the concentration or availability of the amino acid or the charged aminoacylated tRNA would have two effects. First, it would reduce the stores of charged tRNAs available at the start of translation making the transient depletion occur after the translation of only a few repeated codons (represented by the lengthened dark area of the lower box in FIG. 4A). Second, the duration of each pause would be on average greater, since the ribosome would need to wait for the now scarce aminoacylated tRNA. The probability of mRNA degradation increases with a greater probability and duration of each pause. These features have been mathematically represented in FIG. 4B by sigmoid equations of the type Y=(1−1/(1+e^{-x})). A left-shift due to deprivation of glutamine is indicated to take into account the enhanced probability of a pause earlier in the repeat. A lower plateau indicates where consumption due to translation (slowed by pausing) equals production by glutaminyl tRNA synthetase activity (whose activity may be reduced by inhibiting the enzyme or by depriving the enzyme of its substrate, glutamine). Several functions other than the sigmoid curve would also be suitable to describe a hypothetical reduction of mRNA stability caused by pausing due to limiting charged glutamine-tRNA. Steady state mRNA levels are typically represented by the kinetic equation K_{TS}=K_{D}*X where K_{TS} is the rate of transcription, K_{D} the normal rate of mRNA decay and X the concentration of the RNA at steady state. Added to this equation is a rate of destruction due to pausing during translation (K_{TD}) to yield the equation K_{TS}=K_{D}*X+K_{TD}*X. Solving for X gives X=K_{TS}/(K_{D}+K_{TD}). For a gene with no clustered glutamine codons K_{TD}=0 and its steady state level, X₀=K_{TS}/K_{D}. Thus the ratio of mRNA concentrations for a gene with repeats (X_{R}) to the concentration without repeats (X₀) is X_{R}/X₀=K_{D}/(K_{D}+K_{TD}). K_{TD}=Ki*P_{D}, where Kᵢ=the rate of translation initiation and P_{D}=the probability that degradation occurs due to pausing during a single translation of the repeat. P_{D}=1−IIP_{S} where IIP_{S} is the product of the probabilities of mRNA survival at each codon of the repeat (shown in panel b). Thus the final equation is X_{R}/X₀=1/(1+(Kᵢ/K_{D})*(1−IIP_{S})). Notice that Kᵢ/K_{D}=the average number of times a message is translated. The curves in FIG. 4C were derived from the Ps from FIG. 4B, with the assumption that the number of translations per message is 180 in high glutamine and one half that rate in low glutamine.

This model predicts that even a small decrease in mRNA survival per glutamine translated (to illustrate a decrease of 0.05% was selected) can have profound effects on the mRNA level when a long glutamine repeat is translated. In the illustration presented, mRNAs with the median normal repeat length in the HD locus of 20 would maintain 99% of wild type levels where mRNAs with the median disease length of 44 would be reduced to 60% (arrows in FIG. 4C). Furthermore, the model predicts relatively little effect on mRNAs where glutamines are not clustered in a repeat. This suggests allele specific reduction of gene expression might be achieved by glutamine deprivation or more efficacious derivatives of such a strategy.

There are several known mRNA degradation pathways involving translation. First, there is a system within eukaryotic cells designed to rapidly degrade mRNAs containing premature stop codons (NMD for nonsense-mediated decay, reviewed in (57, 58)). A current model for this system involves the translating ribosome clearing a fully processed transcript of proteins left near exon-exon junctions after splicing. Transcripts with premature stop codons are not fully cleared of these proteins, since the ribosome is released upstream of some of these splice junctions. These proteins then act as a signal for mRNA degradation. One factor involved in surveillance of mRNAs for such splicing-dependent proteins is Upflp (59). Expression of a dominant negative version of the human homolog of Upflp inhibits NMD (60). Translation is also involved in destroying transcripts that lack stop codons, or non-stop mediated decay (61). A current model of non-stop mediated decay (reviewed in (62)) involves a ribosome stalled at the end of a transcript interacting with several exosome accessory proteins (including ski7 and ski2 proteins), followed by exosome mediated degradation of the mRNA. Any cleavage within the coding region of a transcript that is stimulated by pausing at a long CAG repeat would create a non-stop message that might be degraded by this system.

### Alteration in Gene Expression is Dependent on the Presence of a Rare Cluster of Codons

To determine whether the reductions in gene expression observed in FIGS. 2A and 2B was dependent on the total glutamine content of the polypeptide or the presence of a rare cluster of codons, such as an expanded glutamine repeat, the mRNA produced by ES cells expressing HprtQ150 mRNA and wild type Hdh mRNA were compared. The results of glutamine deprivation on the mRNA levels from these two alleles are shown in FIGS. 2A and B. Table 1 shows the distribution of glutamine in HprtQ150 mRNA and wild-type Hdh mRNA. HprtQ150 mRNA comprises a total of: 153 glutamine codons, with 152 glutamines being coded by the CAG codon and 1 glutamine being coded by the CAA codon. HprtQ150 comprises 150 CAG codons in an expanded repeat, with the remaining 3 glutamine codons being distributed along the remainder of the mRNA. In contrast, wild type Hdh mRNA comprises a total of 173 glutamine codons, with 138 glutamines being coded by the CAG codon and 35 glutamines being coded by the CAA codon. However, unlike HprtQ150 mRNA, the longest glutamine coding repeat in the wild type Hdh mRNA is 7 CAG codons.

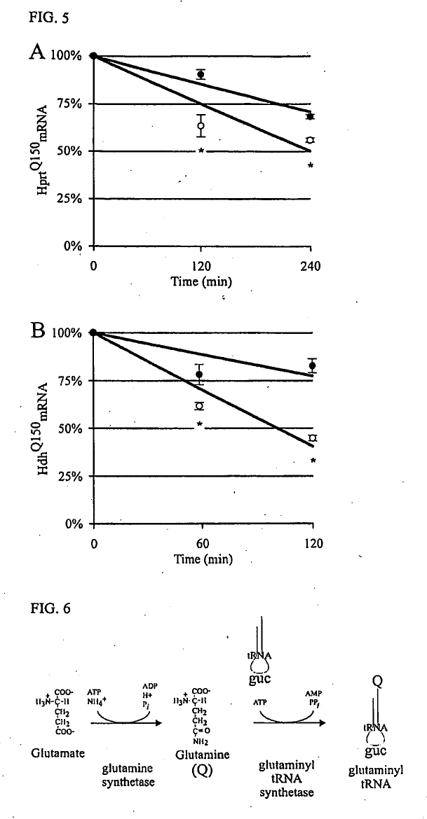

The effect of decreasing glutamine concentration depends on the presence of a rare cluster of codons (in this case a trinucleotide repeat encoding glutamine) rather than the total glutamine content (p=0.0004 Mann-Whitney). Wild type Hdh mRNA has 173 glutamine codons distributed throughout its coding region, yet this mRNA is not susceptible to glutamine deprivation (shown in FIG. 2 B). HprtQ150 mRNA, on the other hand, has only 153 glutamine codons but with a cluster of 150 consecutive glutamine codons and its levels are reduced three fold by glutamine deprivation (shown in FIG. 2 A). This comparison shows that the glutamine codons need to be in a cluster of codons to be susceptible to the effect of glutamine deprivation. Furthermore, the mechanism underlying this decrease involves, at least in part, destabilization of the mRNAs (FIG. 5). FIG. 5 A shows levels of HprtQ150 mRNA at specific times after inhibition of transcription in 1 mg/ml glutamine (filled circles) and 0 mg/ml glutamine media (open circles). Error bars represent the SEM for 9 replicates. Asterisks indicate significant difference between long repeat mRNA levels at 1 and 0 mg/ml glutamine (p<0.0001 Mann-Whitney). FIG. 5 B shows levels of HdhQ150 mRNA at specific times after inhibition of transcription in 1 mg/ml glutamine (filled circles) and 0 mg/ml glutamine media (open circles). Error bars represent the SEM for 9 replicates. Asterisks indicate significant difference between long repeat mRNA levels at 1 and 0 mg/ml glutamine (p<0.0001 Mann-Whitney).

These results are consistent with a model involving reduction of charged glutamine-tRNA levels leading to ribosomal pausing within the repeat followed by destruction of the mRNA.

### Specificity of Alteration of Gene Expression

One major challenge for any potential therapy designed to mimic the amino acid (for example glutamine) deprivation effect (i.e., decreasing the amount of cognate tRNA for the particular codon present in the cluster of codons), is a determination of specificity on gene expression and impact on other cellular processes utilizing the amino acid or the tRNA undergoing deprivation. As an example, consider the effects of glutamine deprivation. Screens for genes with repeat sequences rarely find long CAG repeats. For example, separate screens of cDNA libraries have found only 1 in 2000 and 1 in 7000 cDNAs with CAG repeats longer than 9 CAGs in length (63). Furthermore, a search of the RefSeq database found only 4 of the 19,179 non-redundant human mRNA entries coded for repeats greater than 30 glutamines. None were greater than 40 glutamines in length. Thus repeats of CAG glutamine codons of greater than 30 units in length are rarely found in the genes of the host organism and thus constitute a rare cluster of codons. Furthermore, several of the genes coding for longer repeats were associated with known polyglutamine repeat disorders (e.g. expansions in TATA binding protein which causes SCA17 (19)). Expressed long glutamine repeats might be toxic in general, an idea supported by the Applicants' previous results where ectopic expression of CAG/polyglutamine repeats from a carrier gene caused disease in mice (27). These data leave open the possibility that the side effect of therapies designed to mimic the glutamine deprivation effect to reduce gene expression (reduction of expression of all cellular mRNAs coding for long polyglutamine repeats) will not be toxic. In addition, the results described above indicate that a cluster of codons (in this case a glutamine repeat) is required for effects on gene expression. As discussed above, database screens indicate that no gene or mRNA contained a polyglutamine repeat of over 40 glutamines in length, with most having polyglutamine repeats of 9 glutamines or less. Therefore, the impact of glutamine deprivation therapy on non-specific gene expression is likely to be non-existent or minimal in nature.

The above description is one biological example, where a rare cluster of codons comprising a repeat of a single codon above a certain length creates a sequence that is not found within the coding region of other genes. As discussed previously herein, other examples of rare clusters of codons can be described. For example, the methods described herein also apply to the more complex situation where different codons are present in close proximity within a target gene. In one embodiment, the rare cluster of codons may comprise one or more underrepresented codons. In some cases, a single amino acid is represented by more than one codon. In these cases, the host organism may show a preference for one or more of the codons that specify the same amino acid. For these preferred codons, the amount of cognate tRNA is increased to compensate for the preferred used of the codons. Likewise, for underrepresented codons, the supply of cognate tRNA is reduced. Therefore, the appearance of underrepresented codons provides an opportunity to use the methods of the present disclosure to decrease the expression of genes containing rare clusters of codons comprising one or more underrepresented codons. In calculating a priori, codons that are not frequently used by a host organism would be less likely to be found clustered than codons that are preferred. Such clusters of underrepresented codons are known to be associated with ribosome pause sites with clusters as few as 4 codons. The method described here could be used to decrease the amount of aminoacylated-tRNA of one or more of the underrepresented codons or any codon in close proximity to the underrepresented codons to further exacerbate the pause thus leading to decreased expression of the gene containing such a rare cluster of codons. In an alternate embodiment, the rare cluster of codons may comprise a sequence of codons, that when taken together, appear in no other RNAs or in a few RNAs (i.e., a unique sequence of codons). Therefore, decreasing the levels of the cognate tRNAs to the amino acids represented in such rare cluster of codons will reduce the expression of only those few genes that have the particular rare cluster of codons.

The method described in this disclosure would not need to target a mutation. This principle applies to RNAs with untranslated mutations which cause disease. The levels of RNA containing such untranslated mutations could be altered by the method described here by targeting a rare cluster of codons in the coding region. Furthermore, the method described in this disclosure could target mRNA regions that are not normally translated when used in combination with a treatment that allows translation to continue into the normally untranslated region. This would be particularly useful for diseases such as Myotonic Dystrophy where the repeat is found in the 3′ untranslated region of the mRNA. There are a variety of means whereby translation of normally untranslated regions can be stimulated (e.g. inhibition of splicing or suppression of stop codons) that could be used to make the method described in this disclosure applicable to clusters in normally non-translated regions.

The rare clusters of codons described herein can be identified from private and publicly available databases by techniques known in the art. The completed sequence of the genomes of several potential subject organisms and the computer programs that allow searching for combinations of amino acids or codons in the open reading frames and mRNAs of these organisms are readily available. These resources allow searching for rare clusters of codons, such as but not limited to, trinucleotide repeats and sequences comprising one or more underrepresented codons or combinations of codons that occur in one or a few genes. Examples of such databases and sources of computer programs for searches include public sequence databases such as GenBank, RefSeq and the Swiss Protein database as well as commercial databases and programs such as the ones sold by Celera and the Accelrys.

Specificity by this method might also be influenced by specific features of the target gene. As an example, several underrepresented codons are present in the c-myc transcript and are critical for its degradation via the CRD (coding determinant region) pathway. Ribosome pausing at the underrepresented codons occurs because the cognate aminoacylated tRNA is limiting. This ribosome pause allows endonucleases to destroy the c-myc mRNA. By the method described in this application, ribosome pausing would be increased by further limiting the amount of aminoacylated tRNA for one or more of these underrepresented codons or for any codon in close proximity. The increased pause would further stimulate the destruction of the mRNA. Ribosome pausing may be a general means of destroying mRNA with or without similar CRD regions. Thus specificity could be attained by decreasing combinations of aminoacylated tRNAs that are used in translation of the rare cluster of codons within a transcript that one desires to destroy.

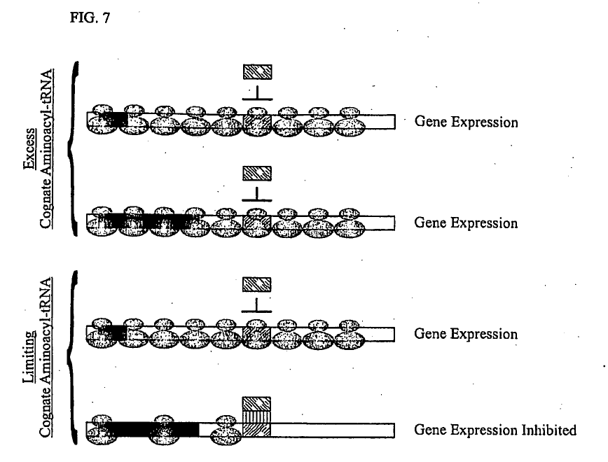

Specificity might also be enhanced by combining sequence specific means of decreasing gene expression (such as RNAi or other methods known in the art) with the amino acid deprivation effect as described herein. The combination of these two methods would increase the amount of reduction and provide enhanced gene or allele specificity, since mRNAs with both the rare cluster of codons (e.g. a trinucleotide repeat) and target of the sequence specific strategy would be more susceptible to reduction in expression levels than other mRNAs. Furthermore, the amino acid deprivation effect would add allele specificity to the sequence specific strategy. Additionally, it would overcome the need for allele specific mutations to occur in one of the few sites susceptible to such sequence based strategies. One possible mechanism of how allele specificity could be conferred when the sequence specific method targets a common sequence in two mRNAs is diagrammed in FIG. 7. In this depiction, the open box represents an mRNA with a mutated site that changes codons within a rare cluster of codons or adds a repeated codon (black box) and a target site for the sequence specific reduction of expression strategy (hatched box). The upper panel represents the conditions normally found in the cell, where the aminoacylated tRNA levels are not limiting for translation elongation and ribosomes inhibit access of molecules designed for sequence specific reduction of expression strategies. When aminoacylated tRNA levels are decreased by the methods described in this disclosure, ribosomes pause allowing access to sites downstream. This combination results in an enhanced quenching of gene expression by the sequence specific method and may provide allele specific reduction of expression when the target of a sequence specific method is common to both allele products. This or another mechanism could provide enhanced or more specific reduction of expression when amino acid deprivation therapy is used in combination with other methods of reducing gene expression. The combination of the effects would provide an additive or synergistic reduction in expression of said target gene. The second means for gene expression may act in an allele specific manner or an allele non-specific manner.

The reduction of expression effect caused by amino acid deprivation could be accomplished in several ways. In one embodiment, this deprivation could be accomplished by lowering the levels of the amino acid itself. The decreased levels of amino acid would, as a result, decrease the levels of the charged tRNA for corresponding amino acid codon or codons. For essential amino acids (those amino acids the body cannot synthesize), such strategies include restricting the dietary intake of the amino acid, inhibition of transport of the amino acid to a particular tissue, stimulating the sequestration of the amino acid from one tissue or compartment at the expense of another, stimulating chemical reactions that lead to a reduction in the levels of the amino acid, stimulating chemical reactions that lead to a reduction in the levels of a precursor of the amino acid, physical methods, such as but not limited to, dialysis to remove the amino acid or a combination of the foregoing. In the case of non-essential amino acids (those amino acids the organism can synthesize) the additional strategies of inhibiting the endogenous synthesis of the amino acid, such as, but not limited to, inhibiting reactions that lead to the synthesis of the amino acid or a precursor to the amino acid, or inhibiting the induction of enzymes needed to produce the amino acid could also be used or combined with the aforementioned methods of amino acid reduction. Furthermore, the effects of amino acid deprivation could be accomplished by inhibiting enzymes involved in the formation of the cognate aminoacylated tRNA (i.e. the tRNA charging reaction).

As one example, potential methods for glutamine deprivation are described. When inhibiting the endogenous synthesis of glutamine, a number of enzymes involved in glutamine biosynthesis may be targeted. In one embodiment, the enzyme glutamine synthetase is targeted. Glutamine synthetase produces glutamine from a glutamate precursor. Several compounds could be used to inhibit glutamine synthetase, including, but not limited to, methionine sulfoximine (MSO), methionine sulfoxide, methionine sulfone, phosphinothricin, 3-amino-3-carboxypropane sulfonamide, serine, 4-N-hydroxyl-L-2,4-diaminobutyric acid, 2-amino-4-phosphobutyric acid, delta-allohydroxylysine and other compounds that may be determined to inhibit glutamine synthetase. In alternate embodiment, compounds known to block the induction of glutamine synthetase that occurs due to natural hormones could be used to decrease overall levels of glutamine synthesis. Such compounds include, but are not limited to selective non-steroidal glucocorticoid receptor antagonists described in (64). Key steps in and modulators of the synthesis of other amino acids may also be targeted in a similar manner.

In an alternate embodiment, the effects of amino acid deprivation could be accomplished by inhibiting enzymes involved in the formation of the translation substrate aminoacylated tRNA (i.e. the tRNA charging reaction). This approach offers the advantage of increased specificity as other cellular pathways that depend on the amino acid for activity will not be impacted.

Again as a non-limiting example, various methods for the inhibition of the aminoacylated-glutaminyl tRNA production are described. Several small molecule inhibitors of aminoacylated-glutaminyl-tRNA synthetase, one of the enzymes involved in the production of the aminoacylated-glutaminyl tRNA, exist and the characteristics for such molecules have been determined (64). Such small molecules may reduce levels of long CAG repeat mRNA without the global effects of glutamine starvation. Suitable small molecule inhibitors include glutaminol, glutaminyl adenylate analogs, 5′-O-[N-(L-glutaminyl)sulfamoyl]adenosine (QSI) other known inhibitors and other compounds that may be identified during screening procedures. Suitable glutaminyl adenylate analogs include, but are not limited to, glutaminol adenylate 5 and 5′-O-[N-(L-glutaminyl)sulfamoyl]adenosines (available from RNA Tech NV, Leuven, Belgium). 5′-O-[N-(L-aminoacyl)sulfamoyl]adenosines have also been used to inhibit alanine, arginine, asparagine, cysteine, glycine, histidine, lysine, proline, serine and threonine tRNA charging reactions. Many of the amino acid alcohols (also know as amino alcohols) are known inhibitors of their corresponding amino acid tRNA synthetases including but not limited to L-leucinol, L-phenylalaminol, L-alaminol, L-histidinol, L-tyrosinol, L-methioninol (65). Analogously, glutaminol is expected to inhibit the glutamine-tRNA synthetase. As discussed above, similar approaches could be used to inhibit the production of other aminoacylated tRNAs. The reactions whose inhibition will reduce levels of charged glutamine-tRNA are shown in FIG. 6. The analogous reactions for other charged-tRNA are also known in the art.

In an alternate embodiment the levels of aminoacylated tRNAs are altered by varying levels of hormones known to increase levels of such tRNAs. For example, antagonists to hydrocortisone induction of Leucyl-tRNA and its synthetases has been shown in (66).

In an alternate embodiment the availability of an amino acid to a desired compartment or location of a subject organism is altered to reduce the local concentration of a specific amino acid in a desired cell type. This could be accomplished by inhibition of transporters used to allow or carry an amino acid into a cell. This could also be achieved by increasing the transport into one organ or compartment at the expense of another location. For example, glutamine transport into the liver is increased by glucagon, insulin, and glucocorticoids (50). Such treatment might lower glutamine concentrations in other areas of the body by sequestration to the liver. Alternately, antagonists to such hormonal action might be used to decrease concentrations in cells utilizing such hormonal systems to stimulate uptake of amino acids.

In an alternate embodiment the concentration of an amino acid is reduced by stimulating chemical reactions that decrease the levels of the amino acid or a precursor of the amino acid. This could be accomplished by hormonal, pharmaceutical, or other treatments known to enhance such reactions, including providing substrates other than the amino acid that would help drive reactions that consumed the amino acid. Such substrates could include, but are not limited to the alpha keto acids which can react with L-glutamine in a transanimation reaction that consumes the glutamine (67).

In an alternate embodiment physical methods, such as but not limited to dialysis, for example of the type used for patients with kidney failure, could be used to lower concentrations of specific amino acids or precursors of specific amino acids.

In another application of the teachings of the present disclosure, expression from target genes could be increased by increasing the availability of one or more amino acids or their cognate aminoacylated tRNAs that occur within a rare cluster of codons. The methods described above for increasing amino acid levels and for increasing levels of aminoacylated tRNAs are known in the art and could be used to allow for increased expression of target genes comprising rare clusters of codons. Decreasing the levels of specific amino acids or decreasing levels of their aminoacylated tRNAs within some rare clusters of codons could also be used to increase the levels of specific target mRNAs. As a non-limiting example of a potential mechanism by which this could be achieved, reduction of charged tRNAs in a rare cluster of codons downstream of an endonuclease cleavage site within an mRNA would result in a ribosomal pause that would leave the upstream regions of the mRNA covered with ribosomes. Thus, reduction of aminoacylated tRNAs in such a rare cluster of codons could render sequences inaccessible to nucleases that would destroy the mRNA. Such methods of increasing gene expression may also be sued in methods of treatment as described below.

### Methods of Treatment

The present disclosure also provides for methods to treat and/or prevent trinucleotide repeat diseases in a subject in need of such treatment or prevention by any intervention that would alter levels of specific aminoacylated tRNAs. The present disclosure also provides for methods to treat or prevent diseases which depend on expression of a gene containing a rare cluster of codons (a target gene) in a subject in need of such treatment or prevention by any intervention that would alter levels of specific aminoacylated tRNAs.

In one embodiment, the teachings of the present disclosure provide for the treatment and/or prevention of a trinucleotide repeat disease in a subject in need of such treatment. A trinucleotide repeat disease includes, but is not limited to, Huntington's disease, spinobulbar muscular atrophy, dentatorubral-pallidoluysian atrophy, and several of the Spinocerebellar ataxia types 1, 2, 3, 6, 7 and 17 (SCAs), oculopharyngeal muscular dystrophy (OPMD), congenital hypoventilation syndrome (CCHS), holoprosencephaly, infantile spasm syndrome, mental retardation, cleidocranial dysplasia (CCD), synpolydactyl), hand-foot-genital syndrome, blepharophimosis/ptosis/epicanthus inversus syndrome (BPEIS) and Pseudoachondroplasia/MED (1). In a specific embodiment, the trinucleotide repeat disease comprises an expanded CAG repeat coding for a polyglutamine tract or a GCG tract coding for a polyalanine tract or a GAC tract coding for an aspartate tract. The expanded trinucleotide repeat may comprise over 5 repeats, over 20 repeats, over 40 repeats, over 60 repeats, over 80 repeats or over 100 repeats.

The method of treatment comprises the steps of identifying a subject in need of such treatment and/or prevention and initiating in said subject an amino acid deprivation therapy. As used in the present disclosure, “amino acid deprivation therapy” means any intervention that alters (i.e. reduces or increases) the availability of a charged tRNA cognate to the trinucleotide repeat. In the case of a reduction in availability, the reduction in availability may be partial. Such amino acid deprivation therapy may include in one embodiment lowering the concentration of the amino acid encoded by the trinucleotide repeat in said subject, thereby reducing the amino acid substrate for the tRNA charging reaction. The amino acid concentration may be lowered by dietary restrictions directed at decreasing or eliminating the consumption of the desired amino acid, inhibiting the endogenous synthesis of the desired amino acid by inhibiting an enzyme involved in amino acid biosynthesis, direct removal of the amino acids by physical methods, such as but not limited to, dialysis, inhibiting the induction of enzymes needed to produce the amino acids, inhibition of transport of the amino acids to a particular tissue, stimulating the sequestration of the amino acids from one tissue or compartment at the expense of another or a combination of the foregoing. Suitable inhibitors for the endogenous synthesis of the amino acid glutamine through the inhibition of glutamine synthetase are described above and include, but are not limited to, methionine sulfoximine (MSO), methionine sulfoxide, methionine sulfone, phosphinothricin, 3-amino-3-carboxypropane sulfonamide, serine, 4-N-hydroxyl-L-2,4-diaminobutyric acid, 2-amino-4-phosphobutyric acid, delta-allohydroxylysine and other compounds that may be determined to inhibit glutamine synthetase. Other suitable inhibitors for the endogenous synthesis of alternate amino acids are known in the art. In an alternate embodiment, amino acid deprivation therapy may comprise specifically lowering the availability of the aminoacylated tRNA molecule which serves as the translation substrate by inhibiting a step in the tRNA charging reaction or by inhibiting pathways that increase the levels of the tRNA molecules. Suitable inhibitors for the tRNA charging reaction are described above for glutamine and include but are not limited to amino alcohols, glutaminyl adenylate analogs (for example, glutaminol, glutaminol adenylate 5 and 5′-O-[N-(L-glutaminyl)sulfamoyl]adenosines (QSI), and other compounds including ones that may be identified during screening procedures. Other suitable inhibitors for lowering the availability of aminoacylated tRNA molecules are known in the art. For the case of increasing the availability of a charged tRNA cognate to the trinucleotide repeat, the conditions described above can be reversed.

Such amino acid deprivation therapy would thereby treat or prevent the trinucleotide repeat disease in said subject. Such treatment and/or prevention may comprise altering (i.e. decreasing or increasing) the levels of expression of the gene involved in such trinucleotide repeat disease, decreasing the stability of the mRNA encoded by the gene involved in such trinucleotide repeat disease or a combination of the foregoing. As discussed above, a decrease in gene expression need not be absolute to provide benefit in the treatment and/or prevention methods disclosed. In one embodiment, gene expression is inhibited at least 5% or greater as compared to the gene expression observed without treatment. Other mechanisms may also be involved in such treatment and/or prevention.

In an alternate embodiment, the teachings of the present disclosure provide for the treatment and/or prevention of a disease which depend on expression of a gene containing a rare cluster of codons in a subject in need of such treatment by amino acid deprivation therapy. The method of treatment comprises the steps of identifying a subject in need of such treatment and/or prevention and initiating in said subject an amino acid deprivation therapy. The term “amino acid deprivation therapy” is as defined above. The alteration in availability may be partial. Such amino acid deprivation therapy may include in one embodiment lowering the concentration of one or more of the amino acids encoded by the rare cluster of codons, thereby reducing the amino acid substrate for the tRNA charging reaction. The amino acid concentration may be lowered by dietary restrictions directed at decreasing or eliminating the consumption of the desired amino acids, inhibiting the endogenous synthesis of the desired amino acids by inhibiting an enzyme involved in amino acid biosynthesis, direct removal of the amino acids by dialysis, inhibiting the induction of enzymes needed to produce the amino acids, inhibition of transport of the amino acids to a particular tissue, stimulating the sequestration of the amino acids from one tissue or compartment at the expense of another or a combination of the foregoing. Suitable inhibitors for the endogenous synthesis of the amino acid will depend on the amino acid encoded by the rare cluster of codons and are known in the art. In an alternate embodiment, amino acid deprivation therapy may comprise specifically lowering the availability of the aminoacylated tRNA molecule which serves as the translation substrate by inhibiting a step in the tRNA charging reaction or by inhibiting pathways that increase the levels of the tRNA molecules. Suitable inhibitors for the above will depend on the amino acid encoded by the rare cluster of codons and are known in the art.

Such amino acid deprivation therapy would thereby treat and/or prevent the disease in said subject. Such treatment may comprise altering (i.e. decreasing or increasing) the levels of expression of a gene involved in the disease, decreasing the stability of an mRNA encoded by the gene involved in the disease or a combination of the foregoing. As discussed above, a decrease in gene expression need not be absolute to provide benefit in the treatment and/or prevention methods disclosed. In one embodiment, gene expression is inhibited at least 5% or greater as compared to the gene expression observed for the wild type gene. Other mechanisms may also be involved in such treatment and/or prevention.

The methods of the treating and/or preventing discussed herein may also comprise further administering of one or more additional therapeutic agents in combination with those molecules described above.

### Pharmaceutical Compositions

The molecules described above for use in amino acid deprivation therapy and treatment/prevention methods described herein may be administered alone or as a pharmaceutical composition formulated by any method known in the art. Certain exemplary methods for preparing the compounds and pharmaceutical compositions are described herein and should not be considered as limiting examples. Furthermore, the compounds or pharmaceutical compositions may be administered to the subject as is known in the art and determined by a healthcare provider. Certain modes of administration are provided herein and should not be considered as limiting examples. Furthermore, the compound or pharmaceutical composition may be administered with other agents in the methods described herein. Such other agents may be agents that increase the activity of the compounds disclosed, such as by limiting the degradation or inactivation of the compounds disclosed or increasing the absorption or activity of the compounds disclosed.

The compounds and pharmaceutical compositions described can be used in the form of a medicinal preparation, for example, in aerosol, solid, semi-solid or liquid form which contains the compounds disclosed as an active ingredient. In addition, the pharmaceutical compositions may be used in an admixture with an appropriate pharmaceutically acceptable carriers. Such pharmaceutically acceptable carriers include, but are not limited to, organic or inorganic carriers, excipients or diluents suitable for pharmaceutical applications. The active ingredient may be compounded, for example, with the usual non-toxic pharmaceutically acceptable carriers, excipients or diluents for tablets, pellets, capsules, inhalants, suppositories, solutions, emulsions, suspensions, aerosols and any other form suitable for use. Pharmaceutically acceptable carriers for use in pharmaceutical compositions are well known in the pharmaceutical field, and are described, for example, in Remington: The Science and Practice of Pharmacy Pharmaceutical Sciences, Lippincott Williams and Wilkins (A. R. Gennaro editor, 20^{th} edition). Such materials are nontoxic to the recipients at the dosages and concentrations employed and include, but are not limited to, water, talc, gum acacia, gelatin, magnesium trisilicate, keratin, colloidal silica, urea, buffers such as phosphate, citrate, acetate and other organic acid salts, antioxidants such as ascorbic acid, low molecular weight (less than about ten residues) peptides such as polyarginine, proteins, such as serum albumin, gelatin, or immunoglobulins, hydrophilic polymers such as polyvinylpyrrolidinone, amino acids such as glycine, glutamic acid, aspartic acid, or arginine, monosaccharides, disaccharides, and other carbohydrates including cellulose or its derivatives, lactose, mannitol, glucose, mannose, dextrins, potato or corn starch or starch paste, chelating agents such as EDTA, sugar alcohols such as mannitol or sorbitol, counterions such as sodium and/or nonionic surfactants such as Tween, Pluronics or polyethyleneglycol. In addition, the pharmaceutical compositions may comprise auxiliary agents, such as, but not limited to, taste-enhancing agents, stabilizing agents, thickening agents, coloring agents and perfumes.

Pharmaceutical compositions may be prepared for storage or administration by mixing a compound of the present disclosure having a desired degree of purity with physiologically acceptable carriers, excipients, stabilizers, auxiliary agents etc. as is known in the pharmaceutical field. Such pharmaceutical compositions may be provided in sustained release or timed release formulations.

The pharmaceutical compositions may be administered orally in solid dosage forms, such as capsules, tablets, and powders, or in liquid dosage forms, such as elixirs, syrups and suspensions. It can also be administered parenterally, in sterile liquid dosage forms. Furthermore, pharmaceutical compositions may be administered parenterally by transmucosal delivery via solid, liquid or aerosol forms of transdermally via a patch mechanism or ointment. Various types of transmucosal administration—include respiratory tract mucosal administration, nasal mucosal administration, oral transmucosal (such as sublingual and buccal) administration and rectal transmucosal administration.

For preparing solid compositions such as, but not limited to, tablets or capsules, the pharmaceutical compositions may be mixed with an appropriate pharmaceutically acceptable carriers, such as conventional tableting ingredients (lactose, sucrose, mannitol, corn starch, potato starch, alginic acid, microcrystalline cellulose, acacia, gelatin, guns, colloidal silicon dioxide, croscarmellose sodium, talc, sorbitol, stearic acid magnesium stearate, calcium stearate, zinc stearate, stearic acid, dicalcium phosphate other excipients, colorants, diluents, buffering agents, disintegrating agents, moistening agents, preservatives, flavoring agents, and pharmacologically compatible carriers) and diluents (including, but not limited to, water, saline or buffering solutions) to form a substantially homogenous composition. The substantially homogenous composition means the components (a compound as described herein and a pharmaceutically acceptable carrier) are dispersed evenly throughout the composition so that the composition may be readily subdivided into equally effective unit dosage forms such as tablets, pills and capsules. The solid compositions described may be coated or otherwise compounded to provide a dosage form affording the advantage of prolonged action. For example, the tablet or pill can comprise an inner dosage an outer dosage component, the latter being in the form of an envelope over the former. The two components can be separated by an enteric layer which serves to resist disintegration in the stomach and permits the inner component to pass intact through the stomach or to be delayed in release. A variety of materials can be used for such enteric layers or coatings such materials including a number of polymeric acids and mixtures of polymeric acids with such materials as shellac, cetyl alcohol and cellulose acetate. The active compounds may also be formulated in rectal compositions such as suppositories or retention enemas, e.g., containing conventional suppository bases such as cocoa butter or other glycerides. The solid compositions may also comprise a capsule, such as hard- or soft-shelled gelatin type containing, for example, surfactants, lubricants, and inert fillers, such as lactose, sucrose, calcium phosphate, and corn starch.

For intranasal administration, intrapulmonary administration or administration by other modes of inhalation, the pharmaceutical compositions may be delivered in the form of a solution or suspension from a pump spray container or as an aerosol spray presentation from a pressurized container or nebulizer, with the use of a suitable propellant (e.g., dichlorodifluoromethane, trichlorofluoromethane, dichlorotetrafluoroethane, nitrogen, propane, carbon dioxide or other suitable gas) or as a dry powder. In the case of an aerosol or dry powder format, the amount (dose) of the compound delivered may be determined by providing a valve to deliver a metered amount.

Liquid forms may be administered orally, parenterally or via transmucosal administration. Suitable forms for liquid administration include aqueous solutions, suitably flavored syrups, aqueous or oil suspensions, and flavored emulsions with edible oils such as cottonseed oil, sesame oil, coconut oil, or peanut oil as well as elixirs and similar pharmaceutical vehicles. Suitable dispersing or suspending agents for aqueous suspensions include synthetic natural gums, such as tragacanth, acacia, alginate, dextran, sodium carboxymethyl cellulose, methylcellulose, polyvinylpyrrolidone or gelatin. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners. Liquid formulations may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. For buccal or sublingual administration, the composition may take the form of tablets or lozenges formulated in conventional manners. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

The compounds disclosed (whether alone or in pharmaceutical compositions) may be formulated for parenteral administration. Parenteral administration includes, but is not limited to, intravenous administration, subcutaneous administration, intramuscular administration, intradermal administration, intrathecal administration, intraarticular administration, intracardiac administration, retrobulbar administration and administration via implants, such as sustained release implants.

The pharmaceutical compositions may be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutically acceptable carriers for injectable compositions are well known to those of ordinary skill in the art. See Pharmaceutics and Pharmacy Practice, J.B. Lippincott Co., Philadelphia, Pa., Banker and Chalmers, Eds., 238-250 (1982) and ASHP Handbook on Injectable Drugs, Toissel, 4th ed., 622-630 (1986).

The pharmaceutical compositions are administered in pharmaceutically effective amount. The pharmaceutically effective amount will, of course, vary depending upon known factors, such as the pharmacodynamic characteristics of the particular compound and its mode and route of administration; the age, health and weight of the subject; the severity and stage of the disease state or condition; the kind of concurrent treatment; the frequency of treatment; and the effect desired. The total amount of the compound administered will also be determined by the route, timing and frequency of administration as well as the existence, nature, and extent of any adverse side effects that might accompany the administration of the compound and the desired physiological effect. It will be appreciated by one skilled in the art that various conditions or disease states, in particular chronic conditions or disease states, may require prolonged treatment involving multiple administrations.

### Methods

Construction of ES Cells and Mouse Models with Long CAG Repeats

The construction of the knock-in mouse lines needed for this proposed work has been described previously(27),(55). The CAG repeat mutations used to make these lines were made in vitro with a technique developed by the Applicants that results in variable length repeats clonable up to 150 CAGs in length(68). For the Hprt locus repeats were inserted into a gene targeting cassette with 12 kilobases of homology to the exon 3 region of the X-linked Hprt locus. Gene targeted ES cells were selected for loss of Hprt function. To construct Hdh variants a repetitive targeting strategy was developed that makes knock-ins to the Hdh locus directly selectable(69).

Since the quantitative real time PCR (QRTPCR) assay (described below) cannot distinguish between different alleles of Hdh it is necessary that the ES cells only express a single allele. For the proposed Hprt knock-ins no extra work was involved, since the mouse Hprt gene is X-linked and there is only one copy of Hprt in each of the male ES cell lines. For Hdh an additional gene targeting reaction is needed to remove the promoter and exon 1 of the wild type copy in the heterozygous long repeat ES cells. The promoter and exon 1 of the wild type Hdh was removed in an ES cell line with an expansion in the other allele, resulting in ES cell lines hemizygous for HdhQ150. These engineered ES cells express only the long repeat version of Hdh and lack wild type Hdh mRNA as determined by RT-PCR across the repeat region.

### Assays of Gene Expression

Several assays can be used for analysis of gene expression. These include western analyses of ES cell lines with Hdh, Hprt and polyglutamine antibodies(27, 55). Monoclonal anti-Hprt and polyclonal anti-Hdh antibodies have been developed by the applicants. In addition, single stranded antisense probes for nuclear run-on experiments have been developed. These include commonly used GAPDH and actin control probes, and two probes each for Hprt mRNA and Hdh mRNA. For both genes one probe is upstream and one downstream of the CAG repeat region to take into account the possibility that transcription across long CAG repeats might be inhibited in a nuclear run-on preparation.

Finally, a quantitative real time PCR (QRTPCR) assays has been developed for both Hprt and Hdh mRNA. These assays involve PCR across the exon2-exon3 junction of Hdh cDNA and the exon7-exon8 junction of Hprt cDNA. Each PCR reaction includes a small oligo containing a quenched fluorescent moiety which binds to DNA between the two PCR primers. Thus the specificity of the reaction is enhanced by both the sequence of the primers and of the probe. During the PCR reaction the polymerase destroys the oligo releasing the quencher to allow fluorescence. Fluorescence is measured though every cycle and the point when it increases beyond threshold is logarithmically related to the amount of starting RNA. The precision of these assays were shown by the linearity of threshold cycle with the log of the sample dilutions (12=0.99). Negative control PCR of cDNA made from cells lacking Hdh or Hprt promoters indicate the reactions are specific to their respective gene products.

The foregoing description illustrates and describes the compounds of the present disclosure. Additionally, the disclosure shows and describes only certain embodiments of the compounds but, as mentioned above, it is to be understood that the teachings of the present disclosure are capable of use in various other combinations, modifications, and environments and is capable of changes or modifications within the scope of the inventive concept as expressed herein, commensurate with the above teachings and/or the skill or knowledge of the relevant art. The embodiments described hereinabove are further intended to explain best modes known of practicing the invention and to enable others skilled in the art to utilize the invention in such, or other, embodiments and with the various modifications required by the particular applications or uses of the invention. Accordingly, the description is not intended to limit the invention to the form disclosed herein. All references cited herein are incorporated by reference as if fully set forth in this disclosure.

### REFERENCES

1. Margolis, R. L., et al., Trinucleotide repeat expansion and neuropsychiatric disease. Arch Gen Psychiatry 56, 1019-31 (1999).
2. Boado, R. J., et al. Antisense-mediated down-regulation of the human huntingtin gene. J Pharmacol Exp Ther 295, 239-43. (2000).
3. Caplen, N. J., et al. Rescue of polyglutamine-mediated cytotoxicity by double-stranded RNA-mediated RNA interference. Hum Mol Genet. 11, 175-84. (2002).
4. Phylactou, L. A., C. Darrah, and M. J. Wood Ribozyme-mediated trans-splicing of a trinucleotide repeat. Nat Genet 18, 378-81. (1998).
5. Yen, L., S. M. Strittmatter, and R. G. Kalb Sequence-specific cleavage of Huntingtin mRNA by catalytic DNA; Ann Neurol 46, 366-73. (1999).
6. Sharp, P. A. RNA interference—2001. Genes Dev 15, 485-90. (2001).
7. Miller, V. M., et al. Allele-specific silencing of dominant disease genes. Proc Natl Acad Sci USA 100, 7195-200 (2003).
8. MacDonald, M. E., et al. The Huntington's disease candidate region exhibits many different haplotypes. Nature Genetics 1, 99-103 (1992).
9. Veyrune, J. L., et al. c-fos mRNA instability determinants present within both the coding and the 3′ non coding region link the degradation of this mRNA to its translation. Oncogene 11, 2127-34 (1995).
10. Lemm, I. and J. Ross Regulation of c-myc mRNA decay by translational pausing in a coding region instability determinant. Mol Cell Biol 22, 3959-69 (2002).
11. Caponigro, G., D. Muhlrad, and R. Parker A small segment of the MAT alpha 1 transcript promotes mRNA decay in Saccharomyces cerevisiae: a stimulatory role for rare codons. Mol Cell Biol 13, 5141-8 (1993).
12. Kabnick, K. S. and D. E. Housman Determinants that contribute to cytoplasmic stability of human c-fos and beta-globin mRNAs are located at several sites in each mRNA. Mol Cell Biol 8, 3244-50 (1988).
13. Yeilding, N. M., M. T. Rehman, and W. M. Lee Identification of sequences in c-myc mRNA that regulate its steady-state levels. Mol Cell Biol 16, 3511-22 (1996).
14. Herrick, D., R. Parker, and A. Jacobson Identification and comparison of stable and unstable mRNAs in Saccharomyces cerevisiae. Mol Cell Biol 10, 2269-84 (1990).
15. Clare, J. J. and S. G. Oliver The regulation of RNA synthesis in yeast. V tRNA charging studies. Mol Gen Genet 188, 96-102 (1982).
16. Lavner, Y. and D. Kotlar Codon bias as a factor in regulating expression via translation rate in the human genome. Gene 2004).
17. Fiers, W. and H. Grosjean On codon usage. Nature 277, 328 (1979).
18. Cummings, C. J. and H. Y. Zoghbi Fourteen and counting: unraveling trinucleotide repeat diseases. Hum Mol Genet 9, 909-16 (2000).
19. Nakamura, K., et al. SCA17, a novel autosomal dominant cerebellar ataxia caused by an expanded polyglutamine in TATA-binding protein. Hum Mol Genet 10, 1441-8 (2001).
20. Quarrell, O. and P. Harper (1991). The clinical neurology of Huntington's disease. Huntington's Disease. P. S. Harper. London, W. B. Saunders Company Ltd. 22: 37-80.
21. Quarrell, O. (1991). The neurobiology of Huntington's Disease. Huntington's Disease. P. S. Harper. London, W. B. Saunders Company Ltd. 22: 141-178.
22. Gusella, J. F., et al. Huntington's Disease. Cold Spring Harbor Symp. 61, 615-626 (1996).
23. Sathasivam, K., et al. Identification of an HD patient with a (CAG) 180 repeat expansion and the propagation of highly expanded CAG repeats in lambda phage. Human Genetics 99, 692-695 (1997).
24. Huntington's Disease Collaborative Research Group A novel gene containing a trinucleotide repeat that is expanded and unstable on Huntington's Disease chromosomes. Cell 72, 971-983 (1993).
25. DiFiglia, M., et al. Aggregation of huntingtin in neuronal intranuclear inclusions and dystrophic neurites in brain. Science 277, 1990-1993 (1997).
26. Maat-Schieman, M. L., et al. Distribution of inclusions in neuronal nuclei and dystrophic neurites in Huntington disease brain. J Neuropathol Exp Neurol 58, 129-137 (1999).
27. Ordway, J. M., et al. Ectopically expressed cag repeats cause intranuclear inclusions and a progressive late onset neurological phenotype in the mouse. Cell 91, 753-763 (1997).
28. Faber, P. W., et al. Huntingtin interacts with a family of WW domain proteins. Hum Mol Genet 7, 1463-74 (1998).
29. Li, X. J., et al. A huntingtin-associated protein-enriched in brain with implications for pathology. Nature 378, 398-402 (1995).
30. Burke, J. R., et al. Huntingtin and DRPLA proteins selectively interact with the enzyme GAPDH. Nature Medicine 2, 347-350 (1996).
31. Kalchman, M. A., et al. HIP1, a human homologue of S. cerevisiae Sla2p, interacts with membrane-associated huntingtin in the brain. Nature Genetics 16, 44-53 (1997).
32. Wanker, E. E., et al. HIP-I: a huntingtin interacting protein isolated by the yeast two-hybrid system. Human Molecular Genetics 6, 487-95 (1997).
33. Huang, C. C., et al. Amyloid formation by mutant huntingtin: threshold, progressivity and recruitment of normal polyglutamine proteins. Somat Cell Mol Genet 24, 217-33 (1998).
34. Panov, A. V., et al. Early mitochondrial calcium defects in Huntington's disease are a direct effect of polyglutamines. Nat Neurosci 5, 731-6. (2002).
35. Cha, J. H., et al. Altered neurotransmitter receptor expression in transgenic mouse models of Huntington's disease. Philosophical Transactions of the Royal Society of London-Series B: Biological Sciences 354, 981-9 (1999).
36. Li, H., et al. Amino-terminal fragments of mutant huntingtin show selective accumulation in striatal neurons and synaptic toxicity. Nature Genetics 25, 385-389 (2000).
37. Gauthier, L. R., et al. Huntingtin controls neurotrophic support and survival of neurons by enhancing BDNF vesicular transport along microtubules. Cell 118, 127-38 (2004).
38. Philips, A. V., L. T. Timchenko, and T. A. Cooper Disruption of splicing regulated by a CUG-binding protein in myotonic dystrophy. Science 280, 737-41 (1998).
39. McLaughlin, B. A., C. Spencer, and J. Eberwine CAG trinucleotide RNA repeats interact with RNA-binding proteins. American Journal of Human Genetics 59, 561-9 (1996).
40. Lin, C.-H., et al. Neurological Abnormalities in a Knock-in Mouse Model of Huntington's Disease. Human Mol. Gen. 10, 137-44 (2001).
41. Reddy, P. H., et al. Behavioural abnormalities and selective neuronal loss in HD transgenic mice expressing mutated full-length HD cDNA. Nat Genet 20, 198-202. (1998).
42. Yamamoto, A., J. J. Lucas, and R. Hen Reversal of neuropathology and motor dysfunction in a conditional model of Huntington's disease. Cell 101, 57-66. (2000).
43. Lin, B., et al. Structural analysis of the 5′ region of mouse and human Huntington disease genes reveals conservation of putative promoter region and di- and trinucleotide polymorphisms. Genomics 25, 707-15 (1995).
44. Duyao, M. P., et al. Inactivation of the mouse Huntington's disease gene homolog Hdh. Science 269, 407-10. (1995).
45. Zeitlin, S., et al. Increased apoptosis and early embryonic lethality in mice nullizygous for the Huntington's disease gene homologue. Nature Genetics 11, 155-63 (1995).
46. White, J. K., et al. Huntingtin is required for neurogenesis and is not impaired by the Huntington's disease CAG expansion. Nat Genet 17, 404-10. (1997).
47. Dietrich, P., et al. Conditional mutagenesis in mice with heat shock promoter-driven cre transgenes. Mamm Genome 11, 196-205 (2000). chimeric mice. J Neurosci 21, 7608-19. (2001).
49. Ambrose, C. M., et al. Structure and expression of the Huntington's Disease gene: Evidence against simple inactivation due to an expanded CAG repeat. Somatic Cell & Mol. Gen. 20, 27-38 (1994).
50. Souba, W. W. (1992). Glutamine: Physiology, Biochemistry and Nutrition in Critical Illness. Austin, R.G. Landes Company.
51. Wilusz, C. J., M. Wormington, and S. W. Peltz The cap-to-tail guide to mRNA turnover. Nat Rev Mol Cell Biol 2, 237-46 (2001).
52. Schwartz, D. C. and R. Parker Mutations in translation initiation factors lead to increased rates of deadenylation and decapping of mRNAs in Saccharomyces cerevisiae. Mol Cell Biol 19, 5247-56 (1999).
53. Aota, S., et al. Codon usage tabulated from the GenBank sequence data. Nucl. Acids Res. 16 Supplement, r315-r319.
54. Schilling, G., et al. Expression of the Huntington's disease (IT15) protein product in HD patients. Hum Mol Genet 4, 1365-71 (1995).
55. Lin, C. H., et al. Neurological abnormalities in a knock-in mouse model of Huntington's disease. Hum Mol Genet 10, 137-44 (2001).
56. Dixon, K. T., et al. Mouse Huntington's disease homolog mRNA levels: variation and allele effects. Gene Expr 11, 221-31 (2004).
57. Hentze, M. W. and A. E. Kulozik A perfect message: RNA surveillance and nonsense-mediated decay. Cell 96, 307-10 (1999).
58. Maquat, L. E. When cells stop making sense: effects of nonsense codons on RNA metabolism in vertebrate cells. Rna 1, 453-65 (1995).
59. Weng, Y., K. Czaplinski, and S. W. Peltz Identification and characterization of mutations in the UPF1 gene that affect nonsense suppression and the formation of the Upf protein complex but not mRNA turnover. Mol Cell Biol 16, 5491-506 (1996).
60. Sun, X., et al. A mutated human homologue to yeast Upf1 protein has a dominant-negative effect on the decay of nonsense-containing mRNAs in mammalian cells. Proc Natl Acad Sci USA 95, 10009-14 (1998).
61. Frischmeyer, P. A., et al. An mRNA surveillance mechanism that eliminates transcripts lacking termination codons. Science 295, 2258-61 (2002).
62. Maquat, L. E. Molecular biology. Skiing toward nonstop mRNA decay. Science 295, 2221-2 (2002).
63. Bulle, F., et al. Identification and chromosomal localization of human genes containing CAG/CTG repeats expressed in testis and brain. Genome Res 7, 705-15 (1997).
64. Einstein, M., et al. Selective glucocorticoid receptor nonsteroidal ligands completely antagonize the dexamethasone mediated induction of enzymes involved in gluconeogenesis and glutamine metabolism. J Steroid Biochem Mol Biol 92, 345-56 (2004).
65. Iiboshi, Y., et al. Amino acid-dependent control of p70(s6k). Involvement of tRNA aminoacylation in the regulation. J Biol Chem 274, 1092-9 (1999).
66. Altman, K., et al. Hydrocortisone induction of rat-liver leucyl-transfer RNA and its synthetases. Proc Natl Acad Sci USA 69, 3567-9 (1972).
67. Cooper, A. J. The role of glutamine transaminase K (GTK) in sulfur and alpha-keto acid metabolism in the brain, and in the possible bioactivation of neurotoxicants. Neurochem Int 44, 557-77 (2004).
68. Ordway, J. M. and P. J. Detloff In vitro synthesis and cloning of long CAG repeats. Biotechniques 21, 609-610 (1996).
69. Cearley, J. A. and P. J. Detloff Efficient repetitive alteration of the mouse Huntington's disease gene by management of background in the tag and exchange gene targeting strategy. Transgenic Research 1-10(2001).

TABLE 1
POSITION AND NUMBER OF GLUTAMINES FROM DIFFERENT ALLELE PRODUCTS

| | | Total | Q codons | | | Longest |
|---|---|---|---|---|---|---|
| Allele | Positions of glutamines in protein | codons | CAG | CAA | Total | Q repeat |
| Hdh | 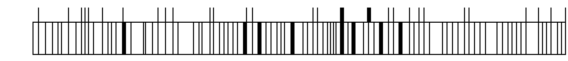 | 3120 | 138 | 35 | 173 | 7 |
| Hdh^{Q150} | 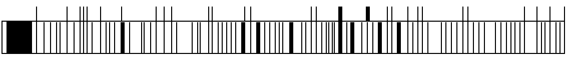 | 3263 | 282 | 34 | 316 | 150 |
| Hprt | 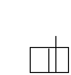 | 219 | 2 | 1 | 3 | 1 |
| hprt^{Q150} | 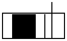 | 380 | 152 | 1 | 153 | 150 |

Protein represented by box with N-terminus on left. Vertical lines reprcscnt glutamine residues with relative positions drawn to scale. Longer lines represent positions of glutamines from the more rarely used CAA codon.

What is claimed:

1. A method of altering the expression of a target gene, said target gene comprising a rare cluster of codons, said method comprising the steps of:
a. identifying said target gene comprising a rare cluster of codons; and
b. modulating the level of at least one aminoacylated tRNA molecule cognate for an amino acid coded for by said rare cluster of codons or modulating the level of at least one amino acid coded for by said rare cluster of codons.

2. The method of claim 1 where said modulating is an increase in the level of said at least one aminoacylated tRNA molecule cognate for an amino acid coded for by said rare cluster of codons or an increase in the level of said at least one amino acid coded for by said rare cluster of codons.

3. The method of claim 2 where said modulating leads to an increase in expression of said target gene.

4. The method of claim 1 where said modulating is a decrease in the level of said at least one aminoacylated tRNA molecule cognate for an amino acid coded for by said rare cluster of codons or a decrease in the level of said at least one amino acid coded for by said rare cluster of codons.

5. The method of claim 4 where said modulating leads to a reduction in expression of said target gene.

6. The method of claim 4 where said modulating leads to an increase in expression of said target gene.

7. The method of claim 5 where said decrease in the level of said at least one amino acid coded for by said rare cluster of codons is accomplished by restricting the dietary intake of said at least one amino acid, inhibiting the transport of said at least one amino acid, stimulating the sequestration of said at least one amino acid, stimulating chemical reaction that lead to a reduction in the level of said at least one amino acid, stimulating chemical reaction that lead to a reduction in the level of a precursor of said at least one amino acid, physical methods that remove said at least one amino acid, inhibiting chemical reactions that lead to the synthesis of said at least one amino acid, inhibiting chemical reactions that lead to the synthesis of a precursor of said at least one amino acid, inhibiting a tRNA charging reaction of said at least one amino acid, or a combination of any of the foregoing.

8. The method of claim 5 where said decrease in the level of said of at least one aminoacylated tRNA molecule cognate for an amino acid coded for by said rare cluster of codons is accomplished by inhibiting the formation of said of at least one aminoacylated tRNA molecules, by altering the levels of hormones that increase the levels of said at least one aminoacylated tRNA molecule, or a combination of the foregoing.

9. The method of claim 1 where said rare cluster of codons comprises at least one underrepresented codon.

10. The method of claim 9 where said at least one underrepresented codon is present in a contiguous sequence of underrepresented codons or a non-contiguous sequence of underrepresented codons.

11. The method of claim 9 where said underrepresented codon is used less than 17 times per 1000 codons or less.

12. The method of claim 1 where said rare cluster of codons comprises a unique sequence of codons.

13. The method of claim 12 where said unique sequence of codons is present in less than about 0.5% of the total mRNAs of said subject, in less than about 1% of the total mRNAs of said subject, in less than about 2.5% of the total mRNAs of said subject or in less than about 5% of the total mRNAs of said subject.

14. The method of claim 1 where said rare cluster of codons comprises a trinucleotide repeat, said trinucleotide repeats having a critical length.

15. The method of claim 14 where said trinucleotide repeat is present in a contiguous sequence or a non-contiguous sequence.

16. The method of claim 14 where said trinucleotide repeat is associated with a trinucleotide repeat disease in said subject.

17. The method of claim 16 where said trinucleotide repeat disease is characterized by a CAG trinucleotide repeat.

18. The method of claim 17 where said modulating is a decrease in the level of said aminoacylated tRNA molecule cognate for said amino acid coded for by said CAG trinucleotide repeat and said decrease leads to a reduction in the expression of said target gene comprising said CAG trinucleotide repeat and a amelioration or prevention of said trinucleotide repeat disease.

19. The method of claim 18 where said trinucleotide repeat disease is selected from the group consisting of Huntington's disease, spinobulbar muscular atrophy, dentatorubral-pallidoluysian atrophy, spinocerebellar ataxia type 1, spinocerebellar ataxia type 2, spinocerebellar ataxia type 3, spinocerebellar ataxia type 6, spinocerebellar ataxia type 7 and spinocerebellar ataxia type 17.

20. The method of claim 18 where said amino acid is a glutamine.

21. The method of claim 17 where said modulating is a decrease in the level of said amino acid coded for by said CAG trinucleotide repeat and said decrease leads to a reduction in the expression of said target gene comprising said CAG trinucleotide repeat and a amelioration or prevention of said trinucleotide repeat disease.

22. The method of claim 21 where said trinucleotide repeat disease is selected from the group consisting of Huntington's disease, spinobulbar muscular atrophy, dentatorubral-pallidoluysian atrophy, spinocerebellar ataxia type 1, spinocerebellar ataxia type 2, spinocerebellar ataxia type 3, spinocerebellar ataxia type 6, spinocerebellar ataxia type 7 and spinocerebellar ataxia type 17.

23. The method of claim 21 where said amino acid is a glutamine.

24. The method of claim 17 where said trinucleotide repeat disease is Huntington's disease, said Huntington's disease characterized by a CAG trinucleotide repeat comprising at least 36 CAG trinucleotide repeats.

25. The method of claim 16 where said trinucleotide repeat disease is characterized by a GCG trinucleotide repeat.

26. The method of claim 25 where said modulating is a decrease in the level of said aminoacylated tRNA molecule cognate for said amino acid coded for by said GCG trinucleotide repeat and said decrease leads to a reduction in the expression of said target gene comprising said GCG trinucleotide repeat and a amelioration or prevention of said trinucleotide repeat disease.

27. The method of claim 26 where said trinucleotide repeat disease is selected from the group consisting of oculopharyngeal muscular dystrophy congenital hypoventilation syndrome, holoprosencephaly, infantile spasm syndrome, mental retardation, X-linked, with isolated growth hormone deficiency, cleidocranial dysplasia, synpolydactyl), hand-foot-genital syndrome, and blepharophimosis/ptosis/epicanthus inversus syndrome.

28. The method of claim 26 where said amino acid is an alanine.

29. The method of claim 25 where said modulating is a decrease in the level of said amino acid coded for by said GCG trinucleotide repeat and said decrease leads to a reduction in the expression of said target gene comprising said GCG trinucleotide repeat and a amelioration or prevention of said trinucleotide repeat disease.

30. The method of claim 29 where said trinucleotide repeat disease is selected from the group consisting of oculopharyngeal muscular dystrophy, congenital hypoventilation syndrome, holoprosencephaly, infantile spasm syndrome, mental retardation, X-linked, with isolated growth hormone deficiency, cleidocranial dysplasia, synpolydactyl), hand-foot-genital syndrome, and blepharophimosis/ptosis/epicanthus inversus syndrome.

31. The method of claim 29 where said amino acid is an alanine.

32. The method of claim 16 where said trinucleotide repeat disease is characterized by a repeat of a GAC trinucleotide repeat.

33. The method of claim 32 where said modulating is a decrease in the level of said aminoacylated tRNA molecule cognate for said amino acid coded for by said GAC trinucleotide repeat and said decrease leads to a reduction in the expression of said target gene comprising said GAC trinucleotide repeat and a amelioration or prevention of said trinucleotide repeat disease.

34. The method of claim 33 where said trinucleotide repeat disease is pseudoachondroplasia/MD.

35. The method of claim 33 where said amino acid is an aspartate.

36. The method of claim 32 where said modulating is a decrease in the level of said amino acid coded for by said GAC trinucleotide repeat and said decrease leads to a reduction in the expression of said target gene comprising said GAC trinucleotide repeat and a amelioration or prevention of said trinucleotide repeat disease.

37. The method of claim 36 where said trinucleotide repeat disease is pseudoachondroplasia/MED.

38. The method of claim 36 where said amino acid is an aspartate.

39. The method of claim 1 where the expression of a single allele of said target gene is altered.

40. The method of claim 1 where the expression both alleles of said target gene are altered.

41. The method of claim 1 further comprising administering a siRNA specific for a portion of said target gene.

42. A method of treating or preventing a disease that depends on the expression of a target gene comprising a rare cluster of codons in a subject in need of said treatment or prevention, said method comprising initiating in said subject an amino acid deprivation therapy so as to alter the level of expression of target gene comprising said rare cluster of codons.

43. The method of claim 42 where said amino acid deprivation therapy decreases the level of at least one aminoacylated tRNA molecule cognate for an amino acid coded for by said rare cluster of codons or decreases the level of at least one amino acid coded for by said rare cluster of codons.

44. The method of claim 43 where said decrease leads to a reduction in expression of said target gene.

45. The method of claim 43 where said decrease leads to an increase in expression of said target gene.

46. The method of claim 44 where said decrease in the level of said of at least one aminoacylated tRNA molecule cognate for an amino acid coded for by said rare cluster of codons is accomplished by inhibiting the formation of said of at least one aminoacylated tRNA molecules, by altering the levels of hormones that increase the levels of said at least one aminoacylated tRNA molecule, or a combination of the foregoing.

47. The method of claim 42 where said amino acid deprivation therapy decreases the level of at least one amino acid coded for by said rare cluster of codons.

48. The method of claim 47 where said decrease leads to a reduction in expression of said target gene.

49. The method of claim 47 where said decrease in the level of said at least one amino acid coded for by said rare cluster of codons is accomplished by restricting the dietary intake of said at least one amino acid, inhibiting the transport of said at least one amino acid, stimulating the sequestration of said at least one amino acid, stimulating chemical reaction that lead to a reduction in the level of said at least one amino acid, stimulating chemical reaction that lead to a reduction in the level of a precursor of said at least one amino acid, physical methods that remove said at least one amino acid, inhibiting chemical reactions that lead to the synthesis of said at least one amino acid, inhibiting chemical reactions that lead to the synthesis of a precursor of said at least one amino acid, inhibiting a tRNA charging reaction of said at least one amino acid, or a combination of any of the foregoing.

50. The method of claim 42 where said rare cluster of codons comprises at least one underrepresented codon.

51. The method of claim 50 where said at least one underrepresented codon is present in a contiguous sequence of underrepresented codons or a non-contiguous sequence of underrepresented codons.

52. The method of claim 50 where said underrepresented codon is used less than 17 times per 1000 codons or less.

53. The method of claim 42 where said rare cluster of codons comprises a unique sequence of codons.

54. The method of claim 53 where said unique sequence of codons is present in less than about 0.5% of the total mRNAs of said subject, in less than about 1% of the total mRNAs of said subject, in less than about 2.5% of the total mRNAs of said subject or in less than about 5% of the total mRNAs of said subject.

55. The method of claim 42 where said rare cluster of codons comprises a trinucleotide repeat, said trinucleotide repeats having a critical length.

56. The method of claim 55 where said trinucleotide repeat is present in a contiguous sequence or a non-contiguous sequence.

57. The method of claim 55 where said trinucleotide repeat is associated with a trinucleotide repeat disease in said subject.

58. The method of claim 55 where said trinucleotide repeat disease is characterized by a. CAG trinucleotide repeat.

59. The method of claim 58 where said modulating is a decrease in the level of said aminoacylated tRNA molecule cognate for said amino acid coded for by said CAG trinucleotide repeat and said decrease leads to a reduction in the expression of said target gene comprising said CAG trinucleotide repeat and a amelioration or prevention of said trinucleotide repeat disease.

60. The method of claim 59 where said trinucleotide repeat disease is selected from the group consisting of Huntington's disease, spinobulbar muscular atrophy, dentatorubral-pallidoluysian atrophy, spinocerebellar ataxia type 1, spinocerebellar ataxia type 2, spinocerebellar ataxia type 3, spinocerebellar ataxia type 6, spinocerebellar ataxia type 7 and spinocerebellar ataxia type 17.

61. The method of claim 59 where said amino acid is a glutamine.

62. The method of claim 58 where said modulating is a decrease in the level of said amino acid coded for by said CAG trinucleotide repeat and said decrease leads to a reduction in the expression of said target gene comprising said CAG trinucleotide repeat and a amelioration or prevention of said trinucleotide repeat disease.

63. The method of claim 62 where said trinucleotide repeat disease is selected from the group consisting of Huntington's disease, spinobulbar muscular atrophy, dentatorubral-pallidoluysian atrophy, spinocerebellar ataxia type 1, spinocerebellar ataxia type 2, spinocerebellar ataxia type 3, spinocerebellar ataxia type 6, spinocerebellar ataxia type 7 and spinocerebellar ataxia type 17.

64. The method of claim 62 where said amino acid is a glutamine.

65. The method of claim 58 where said trinucleotide repeat disease is Huntington's disease, said Huntington's disease characterized by a CAG trinucleotide repeat comprising at least 36 CAG trinucleotide repeats.

66. The method of claim 57 where said trinucleotide repeat disease is characterized by a GCG trinucleotide repeat.

67. The method of claim 66 where said modulating is a decrease in the level of said aminoacylated tRNA molecule cognate for said amino acid coded for by said GCG trinucleotide repeat and said decrease leads to a reduction in the expression of said target gene comprising said GCG trinucleotide repeat and a amelioration or prevention of said trinucleotide repeat disease.

68. The method of claim 67 where said trinucleotide repeat disease is selected from the group consisting of oculopharyngeal muscular dystrophy, congenital hypoventilation syndrome, holoprosencephaly, infantile spasm syndrome, mental retardation, X-linked, with isolated growth hormone deficiency, cleidocranial dysplasia, synpolydactyl), hand-foot-genital syndrome, and blepharophimosis/ptosis/epicanthus inversus syndrome.

69. The method of claim 67 where said amino acid is an alanine.

70. The method of claim 66 where said modulating is a decrease in the level of said amino acid coded for by said GCG trinucleotide repeat and said decrease leads to a reduction in the expression of said target gene comprising said GCG trinucleotide repeat and a amelioration or prevention of said trinucleotide repeat disease.

71. The method of claim 70 where said trinucleotide repeat disease is selected from the group consisting of oculopharyngeal muscular dystrophy, congenital hypoventilation syndrome, holoprosencephaly, infantile spasm syndrome, mental retardation, X-linked, with isolated growth hormone deficiency, cleidocranial dysplasia, synpolydactyl), hand-foot-genital syndrome, and blepharophimosis/ptosis/epicanthus inversus syndrome.

72. The method of claim 70 where said amino acid is an alanine.

73. The method of claim 57 where said trinucleotide repeat disease is characterized by a repeat of a GAC trinucleotide repeat.

74. The method of claim 73 where said modulating is a decrease in the level of said aminoacylated tRNA molecule cognate for said amino acid coded for by said GAC trinucleotide repeat and said decrease leads to a reduction in the expression of said target gene comprising said GAC trinucleotide repeat and a amelioration or prevention of said trinucleotide repeat disease.

75. The method of claim 74 where said trinucleotide repeat disease is pseudoachondroplasia/MED.

76. The method of claim 74 where said amino acid is an aspartate.

77. The method of claim 73 where said modulating is a decrease in the level of said amino acid coded for by said GAC trinucleotide repeat and said decrease leads to a reduction in the expression of said target gene comprising said GAC trinucleotide repeat and a amelioration or prevention of said trinucleotide repeat disease.

78. The method of claim 77 where said trinucleotide repeat disease is pseudoachondroplasia/MED.

79. The method of claim 77 where said amino acid is an aspartate.

80. The method of claim 42 where the expression of a single allele of said target gene is altered.

81. The method of claim 42 where the expression both alleles of said target gene are altered.

82. The method of claim 42 further comprising administering a siRNA specific for a portion of said target gene.

83. A method of altering the expression of a target gene, said target gene comprising a rare cluster of codons, said method comprising the step of modulating the level of at least one aminoacylated tRNA molecule cognate for an amino acid coded for by said rare cluster of codons.

84. The method of claim 83 where said modulating is an increase in the level of said at least one aminoacylated tRNA molecule cognate for an amino acid coded for by said rare cluster of codons.

85. The method of claim 84 where said modulating leads to an increase in expression of said target gene.

86. The method of claim 83 where said modulating is a decrease in the level of said at least one aminoacylated tRNA molecule cognate for an amino acid coded for by said rare cluster of codons.

87. The method of claim 86 where said modulating leads to a reduction in expression of said target gene.

88. The method of claim 86 where said modulating leads to an increase in expression of said target gene.

89. The method of claim 86 where said decrease in the level of said of at least one aminoacylated tRNA molecule cognate for an amino acid coded for by said rare cluster of codons is accomplished by inhibiting the formation of said of at least one aminoacylated tRNA molecules, by altering the levels of hormones that increase the levels of said at least one aminoacylated tRNA molecule, or a combination of the foregoing.

90. The method of claim 89 where said inhibiting the formation of said of at least one aminoacylated tRNA molecules is accomplished, at least in part, by inhibiting a tRNA synthetase enzyme specific for said aminoacylated tRNA molecule.

91. The method of claim 83 where said rare cluster of codons comprises at least one underrepresented codon.

92. The method of claim 90 where said at least one underrepresented codon is present in a contiguous sequence of underrepresented codons or a non-contiguous sequence of underrepresented codons.

93. The method of claim 90 where said underrepresented codon is used less than 17 times per 1000 codons or less.

94. The method of claim 83 where said rare cluster of codons comprises a unique sequence of codons.

95. The method of claim 94 where said unique sequence of codons is present in less than about 0.5% of the total mRNAs of said subject, in less than about 1% of the total mRNAs of said subject, in less than about 2.5% of the total mRNAs of said subject or in less than about 5% of the total mRNAs of said subject.

96. The method of claim 83 where said rare cluster of codons comprises a trinucleotide repeat, said trinucleotide repeats having a critical length.

97. The method of claim 96 where said trinucleotide repeat is present in a contiguous sequence or a non-contiguous sequence.

98. The method of claim 96 where said trinucleotide repeat is associated with a trinucleotide repeat disease in said subject.

99. The method of claim 98 where said trinucleotide repeat disease is characterized by a CAG trinucleotide repeat.

100. The method of claim 99 where said aminoacylated tRNA is an aminoacylated-glutaminyl tRNA and modulating is a decrease in the level of said aminoacylated-glutaminyl tRNA coded for by said CAG trinucleotide repeat and said decrease leads to a reduction in the expression of said target gene comprising said CAG trinucleotide repeat and a amelioration or prevention of said trinucleotide repeat disease.

101. The method of claim 100 where said trinucleotide repeat disease is selected from the group consisting of Huntington's disease, spinobulbar muscular atrophy, dentatorubral-pallidoluysian atrophy, spinocerebellar ataxia type 1, spinocerebellar ataxia type 2, spinocerebellar ataxia type 3, spinocerebellar ataxia type 6, spinocerebellar ataxia type 7 and spinocerebellar ataxia type 17.

102. The method of claim 100 where said decrease in the level of said aminoacylated-glutaminyl tRNA is accomplished by inhibiting the formation of said aminoacylated-glutaminyl tRNA, by altering the levels of hormones that increase the levels of said aminoacylated-glutaminyl tRNA, or a combination of the foregoing.

103. The method of claim 102 where said inhibiting the formation of said aminoacylated-glutaminyl tRNA is accomplished, at least in part, by inhibiting an aminoacylated-glutaminyl-tRNA synthetase.

104. The method of claim 103 where said aminoacylated-glutaminyl-tRNA synthetase is inhibited by a glutaminol, a glutaminyl adenylate analogs, an amino alcohol or a combination of the foregoing.

105. The method of claim 104 where said glutaminyl adenylate analog is selected from the group consisting of glutaminol adenylate 5,5′-O-[N-(L-glutaminyl)sulfamoyl]adenosines and a combination of the foregoing.

106. The method of claim 98 where said trinucleotide repeat disease is Huntington's disease, said Huntington's disease characterized by a CAG trinucleotide repeat comprising at least 36 CAG trinucleotide repeats.

107. The method of claim 106 where said amino acid is a glutamine.

108. The method of claim 98 where said trinucleotide repeat disease is characterized by a GCG trinucleotide repeat.

109. The method of claim 108 where said aminoacylated tRNA is an aminoacylated-alanyl tRNA and said modulating is a decrease in the level of said aminoacylated-alanyl tRNA cognate for said amino acid coded for by said GCG trinucleotide repeat and said decrease leads to a reduction in the expression of said target gene comprising said GCG trinucleotide repeat and a amelioration or prevention of said trinucleotide repeat disease.

110. The method of claim 109 where said trinucleotide repeat disease is selected from the group consisting of oculopharyngeal muscular dystrophy, congenital hypoventilation syndrome, holoprosencephaly, infantile spasm syndrome, mental retardation, X-linked, with isolated growth hormone deficiency, cleidocranial dysplasia, synpolydactyl), hand-foot-genital syndrome, and blepharophimosis/ptosis/epicanthus inversus syndrome.

111. The method of claim 109 where said decrease in the level of said aminoacylated-alanyl tRNA is accomplished by inhibiting the formation of said aminoacylated-alanyl tRNA, by altering the levels of hormones that increase the levels of said aminoacylated-alanyl tRNA, or a combination of the foregoing.

112. The method of claim 111 where said inhibiting the formation of said aminoacylated-alanyl tRNA is accomplished, at least in part, by inhibiting an aminoacylated-alanyl-tRNA synthetase.

113. The method of claim 112 where said aminoacylated-alanyl-tRNA synthetase is inhibited by an alaminol, a glutaminyl adenylate analogs, an amino alcohol or a combination of the foregoing.

114. The method of claim 98 where said trinucleotide repeat disease is characterized by a repeat of a GAC trinucleotide repeat.

115. The method of claim 114 where said aminoacylated tRNA is an aminoacylated-aspartyl tRNA and said modulating is a decrease in the level of said aminoacylated-aspartyl tRNA molecule cognate for said amino acid coded for by said GAC trinucleotide repeat and said decrease leads to a reduction in the expression of said target gene comprising said GAC trinucleotide repeat and a amelioration or prevention of said trinucleotide repeat disease.

116. The method of claim 114 where said trinucleotide repeat disease is pseudoachondroplasia/MED.

117. The method of claim 114 where said decrease in the level of said aminoacylated-aspartyl tRNA is accomplished by inhibiting the formation of said aminoacylated-aspartyl tRNA, by altering the levels of hormones that increase the levels of said aminoacylated-aspartyl tRNA, or a combination of the foregoing.

118. The method of claim 117 where said inhibiting the formation of said aminoacylated-aspartyl tRNA is accomplished, at least in part, by inhibiting an aminoacylated-aspartyl-tRNA synthetase.

119. The method of claim 118 where said aminoacylated-aspartyl-tRNA synthetase is inhibited by an amino alcohol.

120. The method of claim 98 where the expression of a single allele of said target gene is altered.

121. The method of claim 98 where the expression both alleles of said target gene are altered.

122. The method of claim 98 further comprising administering a siRNA specific for a portion of said target gene.


SEQUENCE LISTING (10 sequences)

SEQ ID NO: 1 - DNA, 13495 bp, Homo sapiens
  CDS (146)..(9574)
gctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccaaagaaagaactttcagctaccaagaaagaccgtgtgaatcattgtctgacaatatgtgaaaacatagtggcacagtctgtcagaaattctccagaatttcagaaacttctgggcatcgctatggaactttttctgctgtgcagtgatgacgcagagtcagatgtcaggatggtggctgacgaatgcctcaacaaagttatcaaagctttgatggattctaatcttccaaggttacagctcgagctctataaggaaattaaaaagaatggtgcccctcggagtttgcgtgctgccctgtggaggtttgctgagctggctcacctggttcggcctcagaaatgcaggccttacctggtgaaccttctgccgtgcctgactcgaacaagcaagagacccgaagaatcagtccaggagaccttggctgcagctgttcccaaaattatggcttcttttggcaattttgcaaatgacaatgaaattaaggttttgttaaaggccttcatagcgaacctgaagtcaagctcccccaccattcggcggacagcggctggatcagcagtgagcatctgccagcactcaagaaggacacaatatttctatagttggctactaaatgtgctcttaggcttactcgttcctgtcgaggatgaacactccactctgctgattcttggcgtgctgctcaccctgaggtatttggtgcccttgctgcagcagcaggtcaaggacacaagcctgaaaggcagcttcggagtgacaaggaaagaaatggaagtctctccttctgcagagcagcttgtccaggtttatgaactgacgttacatcatacacagcaccaagaccacaatgttgtgaccggagccctggagctgttgcagcagctcttcagaacgcctccacccgagcttctgcaaaccctgaccgcagtcgggggcattgggcagctcaccgctgctaaggaggagtctggtggccgaagccgtagtgggagtattgtggaacttatagctggagggggttcctcatgcagccctgtcctttcaagaaaacaaaaaggcaaagtgctcttaggagaagaagaagccttggaggatgactctgaatcgagatcggatgtcagcagctctgccttaacagcctcagtgaaggatgagatcagtggagagctggctgcttcttcaggggtttccactccagggtcagcaggtcatgacatcatcacagaacagccacggtcacagcacacactgcaggcggactcagtggatctggccagctgtgacttgacaagctctgccactgatggggatgaggaggatatcttgagccacagctccagccaggtcagcgccgtcccatctgaccctgccatggacctgaatgatgggacccaggcctcgtcgcccatcagcgacagctcccagaccaccaccgaagggcctgattcagctgttaccccttcagacagttctgaaattgtgttagacggtaccgacaaccagtatttgggcctgcagattggacagccccaggatgaagatgaggaagccacaggtattcttcctgatgaagcctcggaggccttcaggaactcttccatggcccttcaacaggcacatttattgaaaaacatgagtcactgcaggcagccttctgacagcagtgttgataaatttgtgttgagagatgaagctactgaaccgggtgatcaagaaaacaagccttgccgcatcaaaggtgacattggacagtccactgatgatgactctgcacctcttgtccattgtgtccgccttttatctgcttcgtttttgctaacagggggaaaaaatgtgctggttccggacagggatgtgagggtcagcgtgaaggccctggccctcagctgtgtgggagcagctgtggccctccacccggaatctttcttcagcaaactctataaagttcctcttgacaccacggaataccctgaggaacagtatgtctcagacatcttgaactacatcgatcatggagacccacaggttcgaggagccactgccattctctgtgggaccctcatctgctccatcctcagcaggtcccgcttccacgtgggagattggatgggcaccattagaaccctcacaggaaatacattttctttggcggattgcattcctttgctgcggaaaacactgaaggatgagtcttctgttacttgcaagttagcttgtacagctgtgaggaactgtgtcatgagtctctgcagcagcagctacagtgagttaggactgcagctgatcatcgatgtgctgactctgaggaacagttcctattggctggtgaggacagagcttctggaaacccttgcagagattgacttcaggctggtgagctttttggaggcaaaagcagaaaacttacacagaggggctcatcattatacagggcttttaaaactgcaagaacgagtgctcaataatgttgtcatccatttgcttggagatgaagaccccagggtgcgacatgttgccgcagcatcactaattaggcttgtcccaaagctgttttataaatgtgaccaaggacaagctgatccagtagtggccgtggcaagagatcaaagcagtgtttacctgaaacttctcatgcatgagacgcagcctccatctcatttctccgtcagcacaataaccagaatatatagaggctataacctactaccaagcataacagacgtcactatggaaaataacctttcaagagttattgcagcagtttctcatgaactaatcacatcaaccaccagagcactcacatttggatgctgtgaagctttgtgtcttctttccactgccttcccagtttgcatttggagtttaggttggcactgtggagtgcctccactgagtgcctcagatgagtctaggaagagctgtaccgttgggatggccacaatgattctgaccctgctctcgtcagcttggttcccattggatctctcagcccatcaagatgctttgattttggccggaaacttgcttgcagccagtgctcccaaatctctgagaagttcatgggcctctgaagaagaagccaacccagcagccaccaagcaagaggaggtctggccagccctgggggaccgggccctggtgcccatggtggagcagctcttctctcacctgctgaaggtgattaacatttgtgcccacgtcctggatgacgtggctcctggacccgcaataaaggcagccttgccttctctaacaaaccccccttctctaagtcccatccgacgaaaggggaaggagaaagaaccaggagaacaagcatctgtaccgttgagtcccaagaaaggcagtgaggccagtgcagcttctagacaatctgatacctcaggtcctgttacaacaagtaaatcctcatcactggggagtttctatcatcttccttcatacctcaaactgcatgatgtcctgaaagctacacacgctaactacaaggtcacgctggatcttcagaacagcacggaaaagtttggagggtttctccgctcagccttggatgttctttctcagatactagagctggccacactgcaggacattgggaagtgtgttgaagagatcctaggatacctgaaatcctgctttagtcgagaaccaatgatggcaactgtttgtgttcaacaattgttgaagactctctttggcacaaacttggcctcccagtttgatggcttatcttccaaccccagcaagtcacaaggccgagcacagcgccttggctcctccagtgtgaggccaggcttgtaccactactgcttcatggccccgtacacccacttcacccaggccctcgctgacgccagcctgaggaacatggtgcaggcggagcaggagaacgacacctcgggatggtttgatgtcctccagaaagtgtctacccagttgaagacaaacctcacgagtgtcacaaagaaccgtgcagataagaatgctattcataatcacattcgtttgtttgaacctcttgttataaaagctttaaaacagtacacgactacaacatgtgtgcagttacagaagcaggttttagatttgctggcgcagctggttcagttacgggttaattactgtcttctggattcagatcaggtgtttattggctttgtattgaaacagtttgaatacattgaagtgggccagttcagggaatcagaggcaatcattccaaacatctttttcttcttggtattactatcttatgaacgctatcattcaaaacagatcattggaattcctaaaatcattcagctctgtgatggcatcatggccagtggaaggaaggctgtgacacatgccataccggctctgcagcccatagtccacgacctctttgtattaagaggaacaaataaagctgatgcaggaaaagagcttgaaacccaaaaagaggtggtggtgtcaatgttactgagactcatccagtaccatcaggtgttggagatgttcattcttgtcctgcagcagtgccacaaggagaatgaagacaagtggaagcgactgtctcgacagatagctgacatcatcctcccaatgttagccaaacagcagatgcacattgactctcatgaagcccttggagtgttaaatacattatttgagattttggccccttcctccctccgtccggtagacatgcttttacggagtatgttcgtcactccaaacacaatggcgtccgtgagcactgttcaactgtggatatcgggaattctggccattttgagggttctgatttcccagtcaactgaagatattgttctttctcgtattcaggagctctccttctctccgtatttaatctcctgtacagtaattaataggttaagagatggggacagtacttcaacgctagaagaacacagtgaagggaaacaaataaagaatttgccagaagaaacattttcaaggtttctattacaactggttggtattcttttagaagacattgttacaaaacagctgaaggtggaaatgagtgagcagcaacatactttctattgccaggaactaggcacactgctaatgtgtctgatccacatcttcaagtctggaatgttccggagaatcacagcagctgccactaggctgttccgcagtgatggctgtggcggcagtttctacaccctggacagcttgaacttgcgggctcgttccatgatcaccacccacccggccctggtgctgctctggtgtcagatactgctgcttgtcaaccacaccgactaccgctggtgggcagaagtgcagcagaccccgaaaagacacagtctgtccagcacaaagttacttagtccccagatgtctggagaagaggaggattctgacttggcagccaaacttggaatgtgcaatagagaaatagtacgaagaggggctctcattctcttctgtgattatgtctgtcagaacctccatgactccgagcacttaacgtggctcattgtaaatcacattcaagatctgatcagcctttcccacgagcctccagtacaggacttcatcagtgccgttcatcggaactctgctgccagcggcctgttcatccaggcaattcagtctcgttgtgaaaacctttcaactccaaccatgctgaagaaaactcttcagtgcttggaggggatccatctcagccagtcgggagctgtgctcacgctgtatgtggacaggcttctgtgcacccctttccgtgtgctggctcgcatggtcgacatccttgcttgtcgccgggtagaaatgcttctggctgcaaatttacagagcagcatggcccagttgccaatggaagaactcaacagaatccaggaataccttcagagcagcgggctcgctcagagacaccaaaggctctattccctgctggacaggtttcgtctctccaccatgcaagactcacttagtccctctcctccagtctcttcccacccgctggacggggatgggcacgtgtcactggaaacagtgagtccggacaaagactggtacgttcatcttgtcaaatcccagtgttggaccaggtcagattctgcactgctggaaggtgcagagctggtgaatcggattcctgctgaagatatgaatgccttcatgatgaactcggagttcaacctaagcctgctagctccatgcttaagcctagggatgagtgaaatttctggtggccagaagagtgccctttttgaagcagcccgtgaggtgactctggcccgtgtgagcggcaccgtgcagcagctccctgctgtccatcatgtcttccagcccgagctgcctgcagagccggcggcctactggagcaagttgaatgatctgtttggggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgaggccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccgttgcagtgcagcctggagagcagcttcttagtccagaaagaaggacaaataccccaaaagccatcagcgaggaggaggaggaagtagatccaaacacacagaatcctaagtatatcactgcagcctgtgagatggtggcagaaatggtggagtctctgcagtcggtgttggccttgggtcataaaaggaatagcggcgtgccggcgtttctcacgccattgctcaggaacatcatcatcagcctggcccgcctgccccttgtcaacagctacacacgtgtgcccccactggtgtggaagcttggatggtcacccaaaccgggaggggattttggcacagcattccctgagatccccgtggagttcctccaggaaaaggaagtctttaaggagttcatctaccgcatcaacacactaggctggaccagtcgtactcagtttgaagaaacttgggccaccctccttggtgtcctggtgacgcagcccctcgtgatggagcaggaggagagcccaccagaagaagacacagagaggacccagatcaacgtcctggccgtgcaggccatcacctcactggtgctcagtgcaatgactgtgcctgtggccggcaacccagctgtaagctgcttggagcagcagccccggaacaagcctctgaaagctctcgacaccaggtttgggaggaagctgagcattatcagagggattgtggagcaagagattcaagcaatggtttcaaagagagagaatattgccacccatcatttatatcaggcatgggatcctgtcccttctctgtctccggctactacaggtgccctcatcagccacgagaagctgctgctacagatcaaccccgagcgggagctggggagcatgagctacaaactcggccaggtgtccatacactccgtgtggctggggaacagcatcacacccctgagggaggaggaatgggacgaggaagaggaggaggaggccgacgcccctgcaccttcgtcaccacccacgtctccagtcaactccaggaaacaccgggctggagttgacatccactcctgttcgcagtttttgcttgagttgtacagccgctggatcctgccgtccagctcagccaggaggaccccggccatcctgatcagtgaggtggtcagatcccttctagtggtctcagacttgttcaccgagcgcaaccagtttgagctgatgtatgtgacgctgacagaactgcgaagggtgcacccttcagaagacgagatcctcgctcagtacctggtgcctgccacctgcaaggcagctgccgtccttgggatggacaaggccgtggcggagcctgtcagccgcctgctggagagcacgctcaggagcagccacctgcccagcagggttggagccctgcacggcgtcctctatgtgctggagtgcgacctgctggacgacactgccaagcagctcatcccggtcatcagcgactatctcctctccaacctgaaagggatcgcccactgcgtgaacattcacagccagcagcacgtactggtcatgtgtgccactgcgttttacctcattgagaactatcctctggacgtagggccggaattttcagcatcaataatacagatgtgtggggtgatgctgtctggaagtgaggagtccaccccctccatcatttaccactgtgccctcagaggcctggagcgcctcctgctctctgagcagctctcccgcctggatgcagaatcgctggtcaagctgagtgtggacagagtgaacgtgcacagcccgcaccgggccatggcggctctgggcctgatgctcacctgcatgtacacaggaaaggagaaagtcagtccgggtagaacttcagaccctaatcctgcagcccccgacagcgagtcagtgattgttgctatggagcgggtatctgttctttttgataggatcaggaaaggctttccttgtgaagccagagtggtggccaggatcctgccccagtttctagacgacttcttcccaccccaggacatcatgaacaaagtcatcggagagtttctgtccaaccagcagccatacccccagttcatggccaccgtggtgtataaggtgtttcagactctgcacagcaccgggcagtcgtccatggtccgggactgggtcatgctgtccctctccaacttcacgcagagggccccggtcgccatggccacgtggagcctctcctgcttctttgtcagcgcgtccaccagcccgtgggtcgcggcgatcctcccacatgtcatcagcaggatgggcaagctggagcaggtggacgtgaaccttttctgcctggtcgccacagacttctacagacaccagatagaggaggagctcgaccgcagggccttccagtctgtgcttgaggtggttgcagccccaggaagcccatatcaccggctgctgacttgtttacgaaatgtccacaaggtcaccacctgctgagcgccatggtgggagagactgtgaggcggcagctggggccggagcctttggaagtctgcgcccttgtgccctgcctccaccgagccagcttggtccctatgggcttccgcacatgccgcgggcggccaggcaacgtgcgtgtctctgccatgtggcagaagtgctctttgtggcagtggccaggcagggagtgtctgcagtcctggtggggctgagcctgaggccttccagaaagcaggagcagctgtgctgcaccccatgtgggtgaccaggtcctttctcctgatagtcacctgctggttgttgccaggttgcagctgctcttgcatctgggccagaagtcctccctcctgcaggctggctgttggcccctctgctgtcctgcagtagaaggtgccgtgagcaggctttgggaacactggcctgggtctccctggtggggtgtgcatgccacgccccgtgtctggatgcacagatgccatggcctgtgctgggccagtggctgggggtgctagacacccggcaccattctcccttctctcttttcttctcaggatttaaaatttaattatatcagtaaagagattaattttaacgtaactctttctatgcccgtgtaaagtatgtgaatcgcaaggcctgtgctgcatgcgacagcgtccggggtggtggacagggcccccggccacgctccctctcctgtagccactggcatagccctcctgagcacccgctgacatttccgttgtacatgttcctgtttatgcattcacaaggtgactgggatgtagagaggcgttagtgggcaggtggccacagcaggactgaggacaggcccccattatcctaggggtgcgctcacctgcagcccctcctcctcgggcacagacgactgtcgttctccacccaccagtcagggacagcagcctccctgtcactcagctgagaaggccagccctccctggctgtgagcagcctccactgtgtccagagacatgggcctcccactcctgttccttgctagccctggggtggcgtctgcctaggagctggctggcaggtgttgggacctgctgctccatggatgcatgccctaagagtgtcactgagctgtgttttgtctgagcctctctcggtcaacagcaaagcttggtgtcttggcactgttagtgacagagcccagcatcccttctgcccccgttccagctgacatcttgcacggtgaccccttttagtcaggagagtgcagatctgtgctcatcggagactgccccacggccctgtcagagccgccactcctatccccaggccaggtccctggaccagcctcctgtttgcaggcccagaggagccaagtcattaaaatggaagtggattctggatggccgggctgctgctgatgtaggagctggatttgggagctctgcttgccgactggctgtgagacgaggcaggggctctgcttcctcagccctagaggcgagccaggcaaggttggcgactgtcatgtggcttggtttggtcatgcccgtcgatgttttgggtattgaatgtggtaagtggaggaaatgttggaactctgtgcaggtgctgccttgagacccccaagcttccacctgtccctctcctatgtggcagctggggagcagctgagatgtggacttgtatgctgcccacatacgtgagggggagctgaaagggagcccctcctctgagcagcctctgccaggcctgtatgaggcttttcccaccagctcccaacagaggcctcccccagccaggaccacctcgtcctcgtggcggggcagcaggagcggtagaaaggggtccgatgtttgaggaggcccttaagggaagctactgaattataacacgtaagaaaatcaccattcttccgtattggttgggggctcctgtttctcatcctagctttttcctggaaagcccgctagaaggtttgggaacgaggggaaagttctcagaactgttggctgctccccacccgcctcccgcctcccccgcaggttatgtcagcagctctgagacagcagtatcacaggccagatgttgttcctggctagatgtttacatttgtaagaaataacactgtgaatgtaaaacagagccattcccttggaatgcatatcgctgggctcaacatagagtttgtcttcctcttgtttacgacgtgatctaaaccagtccttagcaaggggctcagaacaccccgctctggcagtaggtgtcccccacccccaaagacctgcctgtgtgctccggagatgaatatgagctcattagtaaaaatgacttcacccacgcatatacataaagtatccatgcatgtgcatatagacacatctataattttacacacacacctctcaagacggagatgcatggcctctaagagtgcccgtgtcggttcttcctggaagttgactttccttagacccgccaggtcaagttagccgcgtgacggacatccaggcgtgggacgtggtcagggcagggctcattcattgcccactaggatcccactggcgaagatggtctccatatcagctctctgcagaagggaggaagactttatcatgttcctaaaaatctgtggcaagcacccatcgtattatccaaattttgttgcaaatgtgattaatttggttgtcaagttttgggggtgggctgtggggagattgcttttgttttcctgctggtaatatcgggaaagattttaatgaaaccagggtagaattgtttggcaatgcactgaagcgtgtttctttcccaaaatgtgcctcccttccgctgcgggcccagctgagtctatgtaggtgatgtttccagctgccaagtgctctttgttactgtccaccctcatttctgccagcgcatgtgtcctttcaaggggaaaatgtgaagctgaaccccctccagacacccagaatgtagcatctgagaaggccctgtgccctaaaggacacccctcgcccccatcttcatggagggggtcatttcagagccctcggagccaatgaacagctcctcctcttggagctgagatgagccccacgtggagctcgggacggatagtagacagcaataactcggtgtgtggccgcctggcaggtggaacttcctcccgttgcggggtggagtgaggttagttctgtgtgtctggtgggtggagtcaggcttctcttgctacctgtgagcatccttcccagcagacatcctcatcgggctttgtccctcccccgcttcctccctctgcggggaggacccgggaccacagctgctggccagggtagacttggagctgtcctccagaggggtcacgtgtaggagtgagaagaaggaagatcttgagagctgctgagggaccttggagagctcaggatggctcagacgaggacactcgcttgccgggcctgggcctcctgggaaggagggagctgctcagaatgccgcatgacaactgaaggcaacctggaaggttcaggggccgctcttcccccatgtgcctgtcacgctctggtgcagtcaaaggaacgccttcccctcagttgtttctaagagcagagtctcccgctgcaatctgggtggtaactgccagccttggaggatcgtggccaacgtggacctgcctacggagggtgggctctgacccaagtggggcctccttgtccaggtctcactgctttgcaccgtggtcagagggactgtcagctgagcttgagctcccctggagccagcagggctgtgatgggcgagtcccggagccccacccagacctgaatgcttctgagagcaaagggaaggactgacgagagatgtatatttaattttttaactgctgcaaacattgtacatccaaattaaaggaaaaaaatggaaaccatcaaaaaaaaaaaaaaaaaa

SEQ ID NO: 2 - protein, 3142 aa, Homo sapiens
MATLEKLMKAFESLKSFQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 3 - DNA, 10081 bp, Mus musculus
  CDS (168)..(9530)
gcactcgccgcgagggttgccgggacgggcccaagatggctgagcgccttggttccgcttctgcctgccgcgcagagccccattcattgccttgctgctaagtggcgccgcgtagtgccagtaggctccaagtcttcagggtctgtcccatcgggcaggaagccgtcatggcaaccctggaaaagctgatgaaggctttcgagtcgctcaagtcgtttcagcagcaacagcagcagcagccaccgccgcaggcgccgccgccaccgccgccgccgcctccgcctcaaccccctcagccgccgcctcaggggcagccgccgccgccaccaccgccgctgccaggtccggcagaggaaccgctgcaccgaccaaagaaggaactctcagccaccaagaaagaccgtgtgaatcattgtctaacaatatgtgaaaacattgtggcacagtctctcagaaattctccagaatttcagaaactcttgggcatcgctatggaactgtttctgctgtgcagtgacgatgcggagtcagatgtcagaatggtggctgatgagtgcctcaacaaagtcatcaaagctttgatggattctaatcttccaaggctacagttagaactctataaggaaattaaaaagaatggtgctcctcgaagtttgcgtgctgccctgtggaggtttgctgagctggctcacctggttcgacctcagaagtgcaggccttacctggtgaatcttcttccatgcctgacccgaacaagcaaaagaccggaggaatcagttcaggagaccttggctgcagctgttcctaaaattatggcttcttttggcaatttcgcaaatgacaatgaaattaaggttctgttgaaagctttcatagcaaatctgaagtcaagctctcccaccgtgcggcggacagcagccggctcagccgtgagcatctgccaacattctaggaggacacagtacttctacaactggctccttaatgtcctcctaggtctgctggttcccatggaagaagagcactccactctcctgatcctcggtgtgttgctcacattgaggtgtctagtgcccttgctccagcagcaggtcaaggacacaagtctaaaaggcagctttggggtgacacggaaagaaatggaagtctctccttctacagagcagcttgtccaggtttatgaactgactttgcatcatactcagcaccaagaccacaatgtggtgacaggggcactggagctcctgcagcagctcttccgtacccctccacctgaactcctgcaagcactgaccacaccaggagggcttgggcagctcactctggttcaagaagaggcccggggccgaggccgcagcgggagcatcgtggagcttttagctggagggggttcctcgtgcagccctgtcctctcaagaaagcagaaaggcaaagtgctcttaggagaggaagaagccttggaagatgactcggagtccaggtcagatgtcagcagctcagcctttgcagcctctgtgaagagtgagattggtggagagctcgctgcttcttcaggtgtttccactcctggttctgttggtcacgacatcatcactgagcagcctagatcccagcacacacttcaagcagactctgtggatttgtccggctgtgacctgaccagtgctgctactgatggggatgaggaggacatcttgagccacagctccagccagttcagtgctgtcccatccgaccctgccatggacctgaatgatgggacccaggcctcctcacccatcagtgacagttctcagaccaccactgaaggacctgattcagctgtgactccttcggacagttctgaaattgtgttagatggtgccgatagccagtatttaggcatgcagataggacagccacaggaggacgatgaggagggagctgcaggtgttctttctggtgaagtctcagatgttttcagaaactcttctctggcccttcaacaggcacacttgttggaaagaatgggccatagcaggcagccttccgacagcagtatagataagtatgtaacaagagatgaggttgctgaagccagtgatccagaaagcaagccttgccgaatcaaaggtgacataggacagcctaatgatgatgattctgctcctctggtacattgtgtccgtcttttatctgcttcctttttgttaactggtgaaaagaaagcactggttccagacagagacgtgagagtcagtgtgaaggccctggccctcagctgcattggtgcggctgtggcccttcatccagagtcgttcttcagcagactgtacaaagtacctcttaataccacggaaagtactgaggaacagtatgtttctgacatcttgaactacatcgatcatggagacccacaggtccgaggagctactgccattctctgtgggacccttgtctactccatcctcagtaggtcccgtctccgtgttggtgactggctgggcaacatcagaaccctgacaggaaatacattttctctggtggactgcattcctttactgcagaaaacgttgaaggatgaatcttctgttacttgcaagttggcttgtacagctgtgaggcactgtgtcctgagtctttgcagcagcagctacagtgacttgggattacaactgcttattgatatgctgcctctgaagaacagctcctactggctggtgaggaccgaactgctggacactctggcagagattgacttcaggctcgtgagttttttggaggcaaaagcagaaagtttacaccgaggggctcatcattatacagggtttctaaaactacaagaacgagtactcaataatgtggtcatttatttgcttggagatgaagaccccagggttcgacatgttgctgcaacatcattaacaaggcttgtcccaaagctgttttacaagtgtgaccaaggacaagctgatccagttgtggctgtagcgagggatcagagcagtgtctacctgaagctcctcatgcatgagacccagccaccatcacacttttctgtcagcaccatcaccagaatctatagaggctatagcttactgccaagtataacagatgtcaccatggaaaacaatctctcaagagttgttgccgcagtttctcatgaactcattacgtcaacaacacgggcactcacatttggatgctgtgaagccttgtgtcttctctcagcagcctttccagtttgcacttggagtttaggatggcactgtggagtgcccccactgagtgcctctgatgagtccaggaagagctgcactgttgggatggcctccatgattctcaccttgctttcatcagcttggttcccactggatctctcagcccatcaggatgccttgattttggctggaaacttgctagcagcgagtgcccccaagtctctgagaagttcatggacctctgaagaagaagccaactcagcagccaccagacaggaggaaatctggcctgctctgggggatcggactctagtgcccttggtggagcagcttttctcccacctgctgaaggtgatcaatatctgtgctcatgtcttggacgatgtgactcctggaccagcaatcaaggcagccttgccttctctaacaaaccccccttctctaagtcctattcgacggaaagggaaggagaaagaacctggagaacaagcttctactccaatgagtcccaagaaagttggtgaggccagtgcagcctctcgacaatcagacacctcaggacctgtcacagcaagtaaatcatcctcactggggagtttctaccatctcccctcctacctcaaactgcatgatgtcctgaaagccactcacgccaactataaggtcaccttagatcttcagaacagcactgaaaagtttggggggttcctgcgctctgccttggacgtcctttctcagattctagagctggcgacactgcaggacattggaaagtgtgttgaagaggtccttggatacctgaaatcctgctttagtcgagaaccaatgatggcaactgtctgtgtgcagcagctattgaagactctctttgggacaaacttagcctcacagtttgatggcttatcttccaaccccagcaagtctcagtgccgagctcagcgccttggctcttcaagtgtgaggcccggcttatatcactactgcttcatggcaccatacacgcacttcacacaggccttggctgacgcaagcctgaggaacatggtgcaggcggagcaggagcgtgatgcctcggggtggtttgatgtactccagaaagtgtctgcccaattgaagacgaacctaacaagcgtcacaaagaaccgtgcagataagaatgctattcataatcacattaggttatttgagcctcttgttataaaagcattgaagcagtacaccacgacaacatctgtacaattgcagaagcaggttttggatttgctggcacagctggttcagctacgggtcaattactgtctactggattcagaccaggtgttcatcgggtttgtgctgaagcagtttgagtacattgaagtgggccagttcagggaatcagaggcaattattccaaatatatttttcttcctggtattactgtcttatgagcgctaccattcaaaacagatcattggaattcctaaaatcatccagctgtgtgatggcatcatggccagtggaaggaaggccgttacacatgctatacctgctctgcagcccattgtccatgacctctttgtgttacgaggaacaaataaagctgatgcagggaaagagcttgagacacagaaggaggtggtggtctccatgctgttacgactcatccagtaccatcaggtgctggagatgttcatccttgtcctgcagcagtgccacaaggagaatgaggacaagtggaaacggctctctcggcaggtcgcagacatcatcctgcccatgttggccaagcagcagatgcatattgactctcatgaagcccttggagtgttaaataccttgtttgagattttggctccttcctccctacgtcctgtggacatgcttttgcggagtatgttcatcactccaagcacaatggcatctgtaagcactgtgcagctgtggatatctggaatcctcgccattctgagggttctcatttcccagtcaaccgaggacattgttctttgtcgtattcaggagctctccttctctccacacttgctctcctgtccagtgattaacaggttaaggggtggaggcggtaatgtaacactaggagaatgcagcgaagggaaacaaaagagtttgccagaagatacattctcaaggtttcttttacagctggttggtattcttctagaagacatcgttacaaaacagctcaaagtggacatgagtgaacagcagcatacgttctactgccaagagctaggcacactgctcatgtgtctgatccacatattcaaatctggaatgttccggagaatcacagcagctgccactagactcttcaccagtgatggctgtgaaggcagcttctatactctagagagcctgaatgcacgggtccgatccatggtgcccacgcacccagccctggtactgctctggtgtcagatcctacttctcatcaaccacactgaccaccggtggtgggcagaggtgcagcagacacccaagagacacagtctgtcctgcacgaagtcacttaacccccagaagtctggcgaagaggaggattctggctcggcagctcagctgggaatgtgcaatagagaaatagtgcgaagaggggcccttattctcttctgtgattatgtctgtcagaatctccatgactcagaacacttaacatggctcattgtgaatcacattcaagatctgatcagcttgtctcatgagcctccagtacaagactttattagtgccattcatcgtaattctgcagctagtggtctttttatccaggcaattcagtctcgctgtgaaaatctttcaacgccaaccactctgaagaaaacacttcagtgcttggaaggcatccatctcagccagtctggtgctgtgctcacactatatgtggacaggctcctgggcacccccttccgtgcgctggctcgcatggtcgacaccctggcctgtcgccgggtagaaatgcttttggctgcaaatttacagagcagcatggcccagttgccagaggaggaactaaacagaatccaagaacacctccagaacagtgggcttgcacaaagacaccaaaggctctattcactgctggacagattccgactctctactgtgcaggactcacttagccccttgcccccagtcacttcccacccactggatggggatgggcacacatctctggaaacagtgagtccagacaaagactggtacctccagcttgtcagatcccagtgttggaccagatcagattctgcactgctggaaggtgcagagctggtcaaccgtatccctgctgaagatatgaatgacttcatgatgagctcggagttcaacctaagccttttggctccctgtttaagccttggcatgagcgagattgctaatggccaaaagagtcccctctttgaagcagcccgtggggtgattctgaaccgggtgaccagtgttgttcagcagcttcctgctgtccatcaagtcttccagcccttcctgcctatagagcccacggcctactggaacaagttgaatgatctgcttggtgataccacatcataccagtctctgaccatacttgcccgtgccctggcacagtacctggtggtgctctccaaagtgcctgctcatttgcaccttcctcctgagaaggagggggacacggtgaagtttgtggtaatgacagttgaggccctgtcatggcatttgatccatgagcagatcccactgagtctggacctccaagccgggctagactgctgctgcctggcactacaggtgcctggcctctggggggtgctgtcctccccagagtacgtgactcatgcctgctccctcatccattgtgtgcgattcatcctggaagccattgcagtacaacctggagaccagcttctcggtcctgaaagcaggtcacatactccaagagctgtcagaaaggaggaagtagactcagatatacaaaacctcagtcatgtcacttcggcctgcgagatggtggcagacatggtggaatccctgcagtcagtgctggccttgggccacaagaggaacagcaccctgccttcatttctcacagctgtgctgaagaacattgttatcagtctggcccgactccccctagttaacagctatactcgtgtgcctcctctggtatggaaactcgggtggtcacccaagcctggaggggattttggcacagtgtttcctgagatccctgtagagttcctccaggagaaggagatcctcaaggagttcatctaccgcatcaacaccctagggtggaccaatcgtacccagttcgaagaaacttgggccaccctccttggtgtcctggtgactcagcccctggtgatggaacaggaagagagcccaccagaggaagacacagaaagaacccagatccatgtcctggctgtgcaggccatcacctctctagtgctcagtgcaatgaccgtgcctgtggctggcaatccagctgtaagctgcttggagcaacagccccggaacaagccactgaaggctctcgataccagatttggaagaaagctgagcatgatcagagggattgtagaacaagaaatccaagagatggtttcccagagagagaatactgccactcaccattctcaccaggcgtgggatcctgtcccttctctgttaccagctactacaggtgctcttatcagccatgacaagctgctgctgcagatcaacccagagcgggagccaggcaacatgagctacaagctgggccaggtgtccatacactccgtgtggctgggaaataacatcacacccctgagagaggaggaatgggatgaggaagaagaggaagaaagtgatgtccctgcaccaacgtcaccacctgtgtctccagtcaattccagaaaacaccgtgccggggttgatattcactcctgttcgcagtttctgcttgaattgtacagccgatggatcctgccatccagtgcagccagaaggacccccgtcatcctgatcagtgaagtggttcgatctcttcttgtagtgtcagacttattcaccgaacgtacccagtttgaaatgatgtatctgacgctgacagaactacggagagtgcacccttcagaagatgagatcctcattcagtacctggtgcctgccacctgtaaggcagctgctgtccttggaatggacaaaactgtggcagagccagtcagccgcctactggagagcacactgaggagcagccacctgcccagccagatcggagccctgcacggcatcctctatgtgttggagtgtgacctcttggatgacactgcaaagcagctcattccagttgttagtgactatctgctgtccaacctcaaaggaatagcccactgcgtgaacattcacagccagcagcatgtgctggtaatgtgtgccactgctttctacctgatggaaaactaccctctggatgtgggaccagaattttcagcatctgtgatacagatgtgtggagtaatgctgtctggaagtgaggagtccaccccctccatcatttaccactgtgccctccggggtctggagcggctcctgctgtctgagcagctatctcggctagacacagagtccttggtcaagctaagtgtggacagagtgaatgtacaaagcccacacagggccatggcagccctaggcctgatgctcacctgcatgtacacaggaaaggaaaaagccagtccaggcagagcttctgaccccagccctgctacacctgacagcgagtctgtgattgtagctatggagcgagtgtctgttctctttgataggatccgcaagggatttccctgtgaagccagggttgtggcaaggatcctgcctcagttcctagatgacttctttccacctcaagatgtcatgaacaaagtcattggagagttcctgtccaatcagcagccatacccacagttcatggccactgtagtttacaaggtttttcagactctgcacagtgctgggcagtcatccatggtccgggactgggtcatgctgtccctgtccaacttcacacaaagaactccagttgccatggccatgtggagcctctcctgcttccttgttagcgcatctaccagcccatgggtttctgcgatccttccacatgtcatcagcaggatgggcaaactggaacaggtggatgtgaaccttttctgcctggttgccacagacttctacagacaccagatagaggaggaattcgaccgcagggctttccagtctgtgtttgaggtggtggctgcaccaggaagtccataccacaggctgcttgcttgtttgcaaaatgttcacaaggtcaccacctgctgagtagtgcctgtgggacaaaaggctgaaagaaggcagctgctggggcctgagcctccaggagcctgctccaagcttctgctggggctgccttggccgtgcaggcttccacttgtgtcaagtggacagccaggcaatggcaggagtgctttgcaatgagggctatgcagggaacatgcactatgttggggttgagcctgagtcctgggtcctggcctcgctgcagctggtgacagtgctaggttgaccaggtgtttgtctttttcctagtgttcccctggccatagtcgccaggttgcagctgccctggtatgtggatcagaagtcctagctcttgccagatggttctgagcccgcctgctccactgggctggagagctccctcccacatttacccagtaggcatacctgccacaccagtgtctggacacaaaatgaatggtgtgtggggctgggaactggggctgccaggtgtccagcaccattttcctttctgtgttttcttctcaggagttaaaatttaattatatcagtaaagagattaattttaatgt

SEQ ID NO: 4 - protein, 3120 aa, Mus musculus
MATLEKLMKAFESLKSFQQQQQQQPPPQAPPPPPPPPPPQPPQPPPQGQPPPPPPPLPGPAEEPLHRPKKELSATKKDRVNHCLTICENIVAQSLRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTVRRTAAGSAVSICQHSRRTQYFYNWLLNVLLGLLVPMEEEHSTLLILGVLLTLRCLVPLLQQQVKDTSLKGSFGVTRKEMEVSPSTEQLVQVYELTLHHTQHQDHNVVTGALELLQQLFRTPPPELLQALTTPGGLGQLTLVQEEARGRGRSGSIVELLAGGGSSCSPVLSRKQKGKVLLGEEEALEDDSESRSDVSSSAFAASVKSEIGGELAASSGVSTPGSVGHDIITEQPRSQHTLQADSVDLSGCDLTSAATDGDEEDILSHSSSQFSAVPSDPAMDLNDGTQASSPISDSSQTTTEGPDSAVTPSDSSEIVLDGADSQYLGMQIGQPQEDDEEGAAGVLSGEVSDVFRNSSLALQQAHLLERMGHSRQPSDSSIDKYVTRDEVAEASDPESKPCRIKGDIGQPNDDDSAPLVHCVRLLSASFLLTGEKKALVPDRDVRVSVKALALSCIGAAVALHPESFFSRLYKVPLNTTESTEEQYVSDILNYIDHGDPQVRGATAILCGTLVYSILSRSRLRVGDWLGNIRTLTGNTFSLVDCIPLLQKTLKDESSVTCKLACTAVRHCVLSLCSSSYSDLGLQLLIDMLPLKNSSYWLVRTELLDTLAEIDFRLVSFLEAKAESLHRGAHHYTGFLKLQERVLNNVVIYLLGDEDPRVRHVAATSLTRLVPKLFYKCDQGQADPVVAVARDQSSVYLKLLMHETQPPSHFSVSTITRIYRGYSLLPSITDVTMENNLSRVVAAVSHELITSTTRALTFGCCEALCLLSAAFPVCTWSLGWHCGVPPLSASDESRKSCTVGMASMILTLLSSAWFPLDLSAHQDALILAGNLLAASAPKSLRSSWTSEEEANSAATRQEEIWPALGDRTLVPLVEQLFSHLLKVINICAHVLDDVTPGPAIKAALPSLTNPPSLSPIRRKGKEKEPGEQASTPMSPKKVGEASAASRQSDTSGPVTASKSSSLGSFYHLPSYLKLHDVLKATHANYKVTLDLQNSTEKFGGFLRSALDVLSQILELATLQDIGKCVEEVLGYLKSCFSREPMMATVCVQQLLKTLFGTNLASQFDGLSSNPSKSQCRAQRLGSSSVRPGLYHYCFMAPYTHFTQALADASLRNMVQAEQERDASGWFDVLQKVSAQLKTNLTSVTKNRADKNAIHNHIRLFEPLVIKALKQYTTTTSVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQVADIILPMLAKQQMHIDSHEALGVLNTLFEILAPSSLRPVDMLLRSMFITPSTMASVSTVQLWISGILAILRVLISQSTEDIVLCRIQELSFSPHLLSCPVINRLRGGGGNVTLGECSEGKQKSLPEDTFSRFLLQLVGILLEDIVTKQLKVDMSEQQHTFYCQELGTLLMCLIHIFKSGMFRRITAAATRLFTSDGCEGSFYTLESLNARVRSMVPTHPALVLLWCQILLLINHTDHRWWAEVQQTPKRHSLSCTKSLNPQKSGEEEDSGSAAQLGMCNREIVRRGALILFCDYVCQNLHDSEHLTWLIVNHIQDLISLSHEPPVQDFISAIHRNSAASGLFIQAIQSRCENLSTPTTLKKTLQCLEGIHLSQSGAVLTLYVDRLLGTPFRALARMVDTLACRRVEMLLAANLQSSMAQLPEEELNRIQEHLQNSGLAQRHQRLYSLLDRFRLSTVQDSLSPLPPVTSHPLDGDGHTSLETVSPDKDWYLQLVRSQCWTRSDSALLEGAELVNRIPAEDMNDFMMSSEFNLSLLAPCLSLGMSEIANGQKSPLFEAARGVILNRVTSVVQQLPAVHQVFQPFLPIEPTAYWNKLNDLLGDTTSYQSLTILARALAQYLVVLSKVPAHLHLPPEKEGDTVKFVVMTVEALSWHLIHEQIPLSLDLQAGLDCCCLALQVPGLWGVLSSPEYVTHACSLIHCVRFILEAIAVQPGDQLLGPESRSHTPRAVRKEEVDSDIQNLSHVTSACEMVADMVESLQSVLALGHKRNSTLPSFLTAVLKNIVISLARLPLVNSYTRVPPLVWKLGWSPKPGGDFGTVFPEIPVEFLQEKEILKEFIYRINTLGWTNRTQFEETWATLLGVLVTQPLVMEQEESPPEEDTERTQIHVLAVQAITSLVLSAMTVPVAGNPAVSCLEQQPRNKPLKALDTRFGRKLSMIRGIVEQEIQEMVSQRENTATHHSHQAWDPVPSLLPATTGALISHDKLLLQINPEREPGNMSYKLGQVSIHSVWLGNNITPLREEEWDEEEEEESDVPAPTSPPVSPVNSRKHRAGVDIHSCSQFLLELYSRWILPSSAARRTPVILISEVVRSLLVVSDLFTERTQFEMMYLTLTELRRVHPSEDEILIQYLVPATCKAAAVLGMDKTVAEPVSRLLESTLRSSHLPSQIGALHGILYVLECDLLDDTAKQLIPVVSDYLLSNLKGIAHCVNIHSQQHVLVMCATAFYLMENYPLDVGPEFSASVIQMCGVMLSGSEESTPSIIYHCALRGLERLLLSEQLSRLDTESLVKLSVDRVNVQSPHRAMAALGLMLTCMYTGKEKASPGRASDPSPATPDSESVIVAMERVSVLFDRIRKGFPCEARVVARILPQFLDDFFPPQDVMNKVIGEFLSNQQPYPQFMATVVYKVFQTLHSAGQSSMVRDWVMLSLSNFTQRTPVAMAMWSLSCFLVSASTSPWVSAILPHVISRMGKLEQVDVNLFCLVATDFYRHQIEEEFDRRAFQSVFEVVAAPGSPYHRLLACLQNVHKVTTC

SEQ ID NO: 5 - DNA, 1289 bp, Mus musculus
  CDS (88)..(744)
ttacctcactgctttccggagcggtagcacctcctccgccggcttcctcctcagaccgctttttgccgcgagccgaccggtcccgtcatgccgacccgcagtcccagcgtcgtgattagcgatgatgaaccaggttatgacctagatttgttttgtatacctaatcattatgccgaggatttggaaaaagtgtttattcctcatggactgattatggacaggactgaaagacttgctcgagatgtcatgaaggagatgggaggccatcacattgtggccctctgtgtgctcaaggggggctataagttctttgctgacctgctggattacattaaagcactgaatagaaatagtgatagatccattcctatgactgtagattttatcagactgaagagctactgtaatgatcagtcaacgggggacataaaagttattggtggagatgatctctcaactttaactggaaagaatgtcttgattgttgaagatataattgacactggtaaaacaatgcaaactttgctttccctggttaagcagtacagccccaaaatggttaaggttgcaagcttgctggtgaaaaggacctctcgaagtgttggatacaggccagactttgttggatttgaaattccagacaagtttgttgttggatatgcccttgactataatgagtacttcaggaatttgaatcacgtttgtgtcattagtgaaactggaaaagccaaatacaaagcctaagatgagcgcaagttgaatctgcaaatacgaggagtcctgttgatgttgccagtaaaattagcaggtgttctagtcctgtggccatctgcctagtaaagctttttgcatgaaccttctatgaatgttactgttttatttttagaaatgtcagttgctgcgtccccagacttttgatttgcactatgagcctataggccagcctaccctctggtagattgtcgcttatcttgtaagaaaaacaaatctcttaaattaccacttttaaataataatactgagattgtatctgtaagaaggatttaaagagaagctatattagttttttaattggtattttaatttttatatattcaggagagaaagatgtgattgatattgttaatttagacgagtctgaagctctcgatttcctatcagtaacagcatctaagaggttttgctcagtggaataaacatgtttcagcagtgttggctgtattttcccactttcagtaaatcgttgtcaacagttccttttaaatgcaaataaataaattctaaaaatt

SEQ ID NO: 6 - protein, 218 aa, Mus musculus
MPTRSPSVVISDDEPGYDLDLFCIPNHYAEDLEKVFIPHGLIMDRTERLARDVMKEMGGHHIVALCVLKGGYKFFADLLDYIKALNRNSDRSIPMTVDFIRLKSYCNDQSTGDIKVIGGDDLSTLTGKNVLIVEDIIDTGKTMQTLLSLVKQYSPKMVKVASLLVKRTSRSVGYRPDFVGFEIPDKFVVGYALDYNEYFRNLNHVCVISETGKAKYKA

SEQ ID NO: 7 - DNA, 10510 bp, Mus musculus
gcactcgccgcgagggttgccgggacgggcccaagatggctgagcgccttggttccgcttctgcctgccgcgcagagccccattcattgccttgctgctaagtggcgccgcgtagtgccagtaggctccaagtcttcagggtctgtcccatcgggcaggaagccgtcatggcaaccctggaaaagctgatgaaggctttcgagtcgctcaagtcgtttcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagccaccgccgcaggcgccgccgccaccgccgccgccgcctccgcctcaaccccctcagccgccgcctcaggggcagccgccgccgccaccaccgccgctgccaggtccggcagaggaaccgctgcaccgaccaaagaaggaactctcagccaccaagaaagaccgtgtgaatcattgtctaacaatatgtgaaaacattgtggcacagtctctcagaaattctccagaatttcagaaactcttgggcatcgctatggaactgtttctgctgtgcagtgacgatgcggagtcagatgtcagaatggtggctgatgagtgcctcaacaaagtcatcaaagctttgatggattctaatcttccaaggctacagttagaactctataaggaaattaaaaagaatggtgctcctcgaagtttgcgtgctgccctgtggaggtttgctgagctggctcacctggttcgacctcagaagtgcaggccttacctggtgaatcttcttccatgcctgacccgaacaagcaaaagaccggaggaatcagttcaggagaccttggctgcagctgttcctaaaattatggcttcttttggcaatttcgcaaatgacaatgaaattaaggttctgttgaaagctttcatagcaaatctgaagtcaagctctcccaccgtgcggcggacagcagccggctcagccgtgagcatctgccaacattctaggaggacacagtacttctacaactggctccttaatgtcctcctaggtctgctggttcccatggaagaagagcactccactctcctgatcctcggtgtgttgctcacattgaggtgtctagtgcccttgctccagcagcaggtcaaggacacaagtctaaaaggcagctttggggtgacacggaaagaaatggaagtctctccttctacagagcagcttgtccaggtttatgaactgactttgcatcatactcagcaccaagaccacaatgtggtgacaggggcactggagctcctgcagcagctcttccgtacccctccacctgaactcctgcaagcactgaccacaccaggagggcttgggcagctcactctggttcaagaagaggcccggggccgaggccgcagcgggagcatcgtggagcttttagctggagggggttcctcgtgcagccctgtcctctcaagaaagcagaaaggcaaagtgctcttaggagaggaagaagccttggaagatgactcggagtccaggtcagatgtcagcagctcagcctttgcagcctctgtgaagagtgagattggtggagagctcgctgcttcttcaggtgtttccactcctggttctgttggtcacgacatcatcactgagcagcctagatcccagcacacacttcaagcagactctgtggatttgtccggctgtgacctgaccagtgctgctactgatggggatgaggaggacatcttgagccacagctccagccagttcagtgctgtcccatccgaccctgccatggacctgaatgatgggacccaggcctcctcacccatcagtgacagttctcagaccaccactgaaggacctgattcagctgtgactccttcggacagttctgaaattgtgttagatggtgccgatagccagtatttaggcatgcagataggacagccacaggaggacgatgaggagggagctgcaggtgttctttctggtgaagtctcagatgttttcagaaactcttctctggcccttcaacaggcacacttgttggaaagaatgggccatagcaggcagccttccgacagcagtatagataagtatgtaacaagagatgaggttgctgaagccagtgatccagaaagcaagccttgccgaatcaaaggtgacataggacagcctaatgatgatgattctgctcctctggtacattgtgtccgtcttttatctgcttcctttttgttaactggtgaaaagaaagcactggttccagacagagacgtgagagtcagtgtgaaggccctggccctcagctgcattggtgcggctgtggcccttcatccagagtcgttcttcagcagactgtacaaagtacctcttaataccacggaaagtactgaggaacagtatgtttctgacatcttgaactacatcgatcatggagacccacaggtccgaggagctactgccattctctgtgggacccttgtctactccatcctcagtaggtcccgtctccgtgttggtgactggctgggcaacatcagaaccctgacaggaaatacattttctctggtggactgcattcctttactgcagaaaacgttgaaggatgaatcttctgttacttgcaagttggcttgtacagctgtgaggcactgtgtcctgagtctttgcagcagcagctacagtgacttgggattacaactgcttattgatatgctgcctctgaagaacagctcctactggctggtgaggaccgaactgctggacactctggcagagattgacttcaggctcgtgagttttttggaggcaaaagcagaaagtttacaccgaggggctcatcattatacagggtttctaaaactacaagaacgagtactcaataatgtggtcatttatttgcttggagatgaagaccccagggttcgacatgttgctgcaacatcattaacaaggcttgtcccaaagctgttttacaagtgtgaccaaggacaagctgatccagttgtggctgtagcgagggatcagagcagtgtctacctgaagctcctcatgcatgagacccagccaccatcacacttttctgtcagcaccatcaccagaatctatagaggctatagcttactgccaagtataacagatgtcaccatggaaaacaatctctcaagagttgttgccgcagtttctcatgaactcattacgtcaacaacacgggcactcacatttggatgctgtgaagccttgtgtcttctctcagcagcctttccagtttgcacttggagtttaggatggcactgtggagtgcccccactgagtgcctctgatgagtccaggaagagctgcactgttgggatggcctccatgattctcaccttgctttcatcagcttggttcccactggatctctcagcccatcaggatgccttgattttggctggaaacttgctagcagcgagtgcccccaagtctctgagaagttcatggacctctgaagaagaagccaactcagcagccaccagacaggaggaaatctggcctgctctgggggatcggactctagtgcccttggtggagcagcttttctcccacctgctgaaggtgatcaatatctgtgctcatgtcttggacgatgtgactcctggaccagcaatcaaggcagccttgccttctctaacaaaccccccttctctaagtcctattcgacggaaagggaaggagaaagaacctggagaacaagcttctactccaatgagtcccaagaaagttggtgaggccagtgcagcctctcgacaatcagacacctcaggacctgtcacagcaagtaaatcatcctcactggggagtttctaccatctcccctcctacctcaaactgcatgatgtcctgaaagccactcacgccaactataaggtcaccttagatcttcagaacagcactgaaaagtttggggggttcctgcgctctgccttggacgtcctttctcagattctagagctggcgacactgcaggacattggaaagtgtgttgaagaggtccttggatacctgaaatcctgctttagtcgagaaccaatgatggcaactgtctgtgtgcagcagctattgaagactctctttgggacaaacttagcctcacagtttgatggcttatcttccaaccccagcaagtctcagtgccgagctcagcgccttggctcttcaagtgtgaggcccggcttatatcactactgcttcatggcaccatacacgcacttcacacaggccttggctgacgcaagcctgaggaacatggtgcaggcggagcaggagcgtgatgcctcggggtggtttgatgtactccagaaagtgtctgcccaattgaagacgaacctaacaagcgtcacaaagaaccgtgcagataagaatgctattcataatcacattaggttatttgagcctcttgttataaaagcattgaagcagtacaccacgacaacatctgtacaattgcagaagcaggttttggatttgctggcacagctggttcagctacgggtcaattactgtctactggattcagaccaggtgttcatcgggtttgtgctgaagcagtttgagtacattgaagtgggccagttcagggaatcagaggcaattattccaaatatatttttcttcctggtattactgtcttatgagcgctaccattcaaaacagatcattggaattcctaaaatcatccagctgtgtgatggcatcatggccagtggaaggaaggccgttacacatgctatacctgctctgcagcccattgtccatgacctctttgtgttacgaggaacaaataaagctgatgcagggaaagagcttgagacacagaaggaggtggtggtctccatgctgttacgactcatccagtaccatcaggtgctggagatgttcatccttgtcctgcagcagtgccacaaggagaatgaggacaagtggaaacggctctctcggcaggtcgcagacatcatcctgcccatgttggccaagcagcagatgcatattgactctcatgaagcccttggagtgttaaataccttgtttgagattttggctccttcctccctacgtcctgtggacatgcttttgcggagtatgttcatcactccaagcacaatggcatctgtaagcactgtgcagctgtggatatctggaatcctcgccattctgagggttctcatttcccagtcaaccgaggacattgttctttgtcgtattcaggagctctccttctctccacacttgctctcctgtccagtgattaacaggttaaggggtggaggcggtaatgtaacactaggagaatgcagcgaagggaaacaaaagagtttgccagaagatacattctcaaggtttcttttacagctggttggtattcttctagaagacatcgttacaaaacagctcaaagtggacatgagtgaacagcagcatacgttctactgccaagagctaggcacactgctcatgtgtctgatccacatattcaaatctggaatgttccggagaatcacagcagctgccactagactcttcaccagtgatggctgtgaaggcagcttctatactctagagagcctgaatgcacgggtccgatccatggtgcccacgcacccagccctggtactgctctggtgtcagatcctacttctcatcaaccacactgaccaccggtggtgggcagaggtgcagcagacacccaagagacacagtctgtcctgcacgaagtcacttaacccccagaagtctggcgaagaggaggattctggctcggcagctcagctgggaatgtgcaatagagaaatagtgcgaagaggggcccttattctcttctgtgattatgtctgtcagaatctccatgactcagaacacttaacatggctcattgtgaatcacattcaagatctgatcagcttgtctcatgagcctccagtacaagactttattagtgccattcatcgtaattctgcagctagtggtctttttatccaggcaattcagtctcgctgtgaaaatctttcaacgccaaccactctgaagaaaacacttcagtgcttggaaggcatccatctcagccagtctggtgctgtgctcacactatatgtggacaggctcctgggcacccccttccgtgcgctggctcgcatggtcgacaccctggcctgtcgccgggtagaaatgcttttggctgcaaatttacagagcagcatggcccagttgccagaggaggaactaaacagaatccaagaacacctccagaacagtgggcttgcacaaagacaccaaaggctctattcactgctggacagattccgactctctactgtgcaggactcacttagccccttgcccccagtcacttcccacccactggatggggatgggcacacatctctggaaacagtgagtccagacaaagactggtacctccagcttgtcagatcccagtgttggaccagatcagattctgcactgctggaaggtgcagagctggtcaaccgtatccctgctgaagatatgaatgacttcatgatgagctcggagttcaacctaagccttttggctccctgtttaagccttggcatgagcgagattgctaatggccaaaagagtcccctctttgaagcagcccgtggggtgattctgaaccgggtgaccagtgttgttcagcagcttcctgctgtccatcaagtcttccagcccttcctgcctatagagcccacggcctactggaacaagttgaatgatctgcttggtgataccacatcataccagtctctgaccatacttgcccgtgccctggcacagtacctggtggtgctctccaaagtgcctgctcatttgcaccttcctcctgagaaggagggggacacggtgaagtttgtggtaatgacagttgaggccctgtcatggcatttgatccatgagcagatcccactgagtctggacctccaagccgggctagactgctgctgcctggcactacaggtgcctggcctctggggggtgctgtcctccccagagtacgtgactcatgcctgctccctcatccattgtgtgcgattcatcctggaagccattgcagtacaacctggagaccagcttctcggtcctgaaagcaggtcacatactccaagagctgtcagaaaggaggaagtagactcagatatacaaaacctcagtcatgtcacttcggcctgcgagatggtggcagacatggtggaatccctgcagtcagtgctggccttgggccacaagaggaacagcaccctgccttcatttctcacagctgtgctgaagaacattgttatcagtctggcccgactccccctagttaacagctatactcgtgtgcctcctctggtatggaaactcgggtggtcacccaagcctggaggggattttggcacagtgtttcctgagatccctgtagagttcctccaggagaaggagatcctcaaggagttcatctaccgcatcaacaccctagggtggaccaatcgtacccagttcgaagaaacttgggccaccctccttggtgtcctggtgactcagcccctggtgatggaacaggaagagagcccaccagaggaagacacagaaagaacccagatccatgtcctggctgtgcaggccatcacctctctagtgctcagtgcaatgaccgtgcctgtggctggcaatccagctgtaagctgcttggagcaacagccccggaacaagccactgaaggctctcgataccagatttggaagaaagctgagcatgatcagagggattgtagaacaagaaatccaagagatggtttcccagagagagaatactgccactcaccattctcaccaggcgtgggatcctgtcccttctctgttaccagctactacaggtgctcttatcagccatgacaagctgctgctgcagatcaacccagagcgggagccaggcaacatgagctacaagctgggccaggtgtccatacactccgtgtggctgggaaataacatcacacccctgagagaggaggaatgggatgaggaagaagaggaagaaagtgatgtccctgcaccaacgtcaccacctgtgtctccagtcaattccagaaaacaccgtgccggggttgatattcactcctgttcgcagtttctgcttgaattgtacagccgatggatcctgccatccagtgcagccagaaggacccccgtcatcctgatcagtgaagtggttcgatctcttcttgtagtgtcagacttattcaccgaacgtacccagtttgaaatgatgtatctgacgctgacagaactacggagagtgcacccttcagaagatgagatcctcattcagtacctggtgcctgccacctgtaaggcagctgctgtccttggaatggacaaaactgtggcagagccagtcagccgcctactggagagcacactgaggagcagccacctgcccagccagatcggagccctgcacggcatcctctatgtgttggagtgtgacctcttggatgacactgcaaagcagctcattccagttgttagtgactatctgctgtccaacctcaaaggaatagcccactgcgtgaacattcacagccagcagcatgtgctggtaatgtgtgccactgctttctacctgatggaaaactaccctctggatgtgggaccagaattttcagcatctgtgatacagatgtgtggagtaatgctgtctggaagtgaggagtccaccccctccatcatttaccactgtgccctccggggtctggagcggctcctgctgtctgagcagctatctcggctagacacagagtccttggtcaagctaagtgtggacagagtgaatgtacaaagcccacacagggccatggcagccctaggcctgatgctcacctgcatgtacacaggaaaggaaaaagccagtccaggcagagcttctgaccccagccctgctacacctgacagcgagtctgtgattgtagctatggagcgagtgtctgttctctttgataggatccgcaagggatttccctgtgaagccagggttgtggcaaggatcctgcctcagttcctagatgacttctttccacctcaagatgtcatgaacaaagtcattggagagttcctgtccaatcagcagccatacccacagttcatggccactgtagtttacaaggtttttcagactctgcacagtgctgggcagtcatccatggtccgggactgggtcatgctgtccctgtccaacttcacacaaagaactccagttgccatggccatgtggagcctctcctgcttccttgttagcgcatctaccagcccatgggtttctgcgatccttccacatgtcatcagcaggatgggcaaactggaacaggtggatgtgaaccttttctgcctggttgccacagacttctacagacaccagatagaggaggaattcgaccgcagggctttccagtctgtgtttgaggtggtggctgcaccaggaagtccataccacaggctgcttgcttgtttgcaaaatgttcacaaggtcaccacctgctgagtagtgcctgtgggacaaaaggctgaaagaaggcagctgctggggcctgagcctccaggagcctgctccaagcttctgctggggctgccttggccgtgcaggcttccacttgtgtcaagtggacagccaggcaatggcaggagtgctttgcaatgagggctatgcagggaacatgcactatgttggggttgagcctgagtcctgggtcctggcctcgctgcagctggtgacagtgctaggttgaccaggtgtttgtctttttcctagtgttcccctggccatagtcgccaggttgcagctgccctggtatgtggatcagaagtcctagctcttgccagatggttctgagcccgcctgctccactgggctggagagctccctcccacatttacccagtaggcatacctgccacaccagtgtctggacacaaaatgaatggtgtgtggggctgggaactggggctgccaggtgtccagcaccattttcctttctgtgttttcttctcaggagttaaaatttaattatatcagtaaagagattaattttaatgt

SEQ ID NO: 8 - protein, 3263 aa, Mus musculus
MATLEKLMKAFESLKSFQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQPPPQAPPPPPPPPPPQPPQPPPQGQPPPPPPPLPGPAEEPLHRPKKELSATKKDRVNHCLTICENIVAQSLRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTVRRTAAGSAVSICQHSRRTQYFYNWLLNVLLGLLVPMEEEHSTLLILGVLLTLRCLVPLLQQQVKDTSLKGSFGVTRKEMEVSPSTEQLVQVYELTLHHTQHQDHNVVTGALELLQQLFRTPPPELLQALTTPGGLGQLTLVQEEARGRGRSGSIVELLAGGGSSCSPVLSRKQKGKVLLGEEEALEDDSESRSDVSSSAFAASVKSEIGGELAASSGVSTPGSVGHDIITEQPRSQHTLQADSVDLSGCDLTSAATDGDEEDILSHSSSQFSAVPSDPAMDLNDGTQASSPISDSSQTTTEGPDSAVTPSDSSEIVLDGADSQYLGMQIGQPQEDDEEGAAGVLSGEVSDVFRNSSLALQQAHLLERMGHSRQPSDSSIDKYVTRDEVAEASDPESKPCRIKGDIGQPNDDDSAPLVHCVRLLSASFLLTGEKKALVPDRDVRVSVKALALSCIGAAVALHPESFFSRLYKVPLNTTESTEEQYVSDILNYIDHGDPQVRGATAILCGTLVYSILSRSRLRVGDWLGNIRTLTGNTFSLVDCIPLLQKTLKDESSVTCKLACTAVRHCVLSLCSSSYSDLGLQLLIDMLPLKNSSYWLVRTELLDTLAEIDFRLVSFLEAKAESLHRGAHHYTGFLKLQERVLNNVVIYLLGDEDPRVRHVAATSLTRLVPKLFYKCDQGQADPVVAVARDQSSVYLKLLMHETQPPSHFSVSTITRIYRGYSLLPSITDVTMENNLSRVVAAVSHELITSTTRALTFGCCEALCLLSAAFPVCTWSLGWHCGVPPLSASDESRKSCTVGMASMILTLLSSAWFPLDLSAHQDALILAGNLLAASAPKSLRSSWTSEEEANSAATRQEEIWPALGDRTLVPLVEQLFSHLLKVINICAHVLDDVTPGPAIKAALPSLTNPPSLSPIRRKGKEKEPGEQASTPMSPKKVGEASAASRQSDTSGPVTASKSSSLGSFYHLPSYLKLHDVLKATHANYKVTLDLQNSTEKFGGFLRSALDVLSQILELATLQDIGKCVEEVLGYLKSCFSREPMMATVCVQQLLKTLFGTNLASQFDGLSSNPSKSQCRAQRLGSSSVRPGLYHYCFMAPYTHFTQALADASLRNMVQAEQERDASGWFDVLQKVSAQLKTNLTSVTKNRADKNAIHNHIRLFEPLVIKALKQYTTTTSVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQVADIILPMLAKQQMHIDSHEALGVLNTLFEILAPSSLRPVDMLLRSMFITPSTMASVSTVQLWISGILAILRVLISQSTEDIVLCRIQELSFSPHLLSCPVINRLRGGGGNVTLGECSEGKQKSLPEDTFSRFLLQLVGILLEDIVTKQLKVDMSEQQHTFYCQELGTLLMCLIHIFKSGMFRRITAAATRLFTSDGCEGSFYTLESLNARVRSMVPTHPALVLLWCQILLLINHTDHRWWAEVQQTPKRHSLSCTKSLNPQKSGEEEDSGSAAQLGMCNREIVRRGALILFCDYVCQNLHDSEHLTWLIVNHIQDLISLSHEPPVQDFISAIHRNSAASGLFIQAIQSRCENLSTPTTLKKTLQCLEGIHLSQSGAVLTLYVDRLLGTPFRALARMVDTLACRRVEMLLAANLQSSMAQLPEEELNRIQEHLQNSGLAQRHQRLYSLLDRFRLSTVQDSLSPLPPVTSHPLDGDGHTSLETVSPDKDWYLQLVRSQCWTRSDSALLEGAELVNRIPAEDMNDFMMSSEFNLSLLAPCLSLGMSEIANGQKSPLFEAARGVILNRVTSVVQQLPAVHQVFQPFLPIEPTAYWNKLNDLLGDTTSYQSLTILARALAQYLVVLSKVPAHLHLPPEKEGDTVKFVVMTVEALSWHLIHEQIPLSLDLQAGLDCCCLALQVPGLWGVLSSPEYVTHACSLIHCVRFILEAIAVQPGDQLLGPESRSHTPRAVRKEEVDSDIQNLSHVTSACEMVADMVESLQSVLALGHKRNSTLPSFLTAVLKNIVISLARLPLVNSYTRVPPLVWKLGWSPKPGGDFGTVFPEIPVEFLQEKEILKEFIYRINTLGWTNRTQFEETWATLLGVLVTQPLVMEQEESPPEEDTERTQIHVLAVQAITSLVLSAMTVPVAGNPAVSCLEQQPRNKPLKALDTRFGRKLSMIRGIVEQEIQEMVSQRENTATHHSHQAWDPVPSLLPATTGALISHDKLLLQINPEREPGNMSYKLGQVSIHSVWLGNNITPLREEEWDEEEEEESDVPAPTSPPVSPVNSRKHRAGVDIHSCSQFLLELYSRWILPSSAARRTPVILISEVVRSLLVVSDLFTERTQFEMMYLTLTELRRVHPSEDEILIQYLVPATCKAAAVLGMDKTVAEPVSRLLESTLRSSHLPSQIGALHGILYVLECDLLDDTAKQLIPVVSDYLLSNLKGIAHCVNIHSQQHVLVMCATAFYLMENYPLDVGPEFSASVIQMCGVMLSGSEESTPSIIYHCALRGLERLLLSEQLSRLDTESLVKLSVDRVNVQSPHRAMAALGLMLTCMYTGKEKASPGRASDPSPATPDSESVIVAMERVSVLFDRIRKGFPCEARVVARILPQFLDDFFPPQDVMNKVIGEFLSNQQPYPQFMATVVYKVFQTLHSAGQSSMVRDWVMLSLSNFTQRTPVAMAMWSLSCFLVSASTSPWVSAILPHVISRMGKLEQVDVNLFCLVATDFYRHQIEEEFDRRAFQSVFEVVAAPGSPYHRLLACLQNVHKVTTC

SEQ ID NO: 9 - DNA, 1760 bp, Mus musculus
ttacctcactgctttccggagcggtagcacctcctccgccggcttcctcctcagaccgctttttgccgcgagccgaccggtcccgtcatgccgacccgcagtcccagcgtcgtgattagcgatgatgaaccaggttatgacctagatttgttttgtatacctaatcattatgccgaggatttggaaaaagtgtttattcctcatggactgattatggacaggactgaaagacttgctcgacgctggcaccagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcaggtgccgggaagcttcgttagtcgagatgtcatgaaggagatgggaggccatcacattgtggccctctgtgtgctcaaggggggctataagttctttgctgacctgctggattacattaaagcactgaatagaaatagtgatagatccattcctatgactgtagattttatcagactgaagagctactgtaatgatcagtcaacgggggacataaaagttattggtggagatgatctctcaactttaactggaaagaatgtcttgattgttgaagatataattgacactggtaaaacaatgcaaactttgctttccctggttaagcagtacagccccaaaatggttaaggttgcaagcttgctggtgaaaaggacctctcgaagtgttggatacaggccagactttgttggatttgaaattccagacaagtttgttgttggatatgcccttgactataatgagtacttcaggaatttgaatcacgtttgtgtcattagtgaaactggaaaagccaaatacaaagcctaagatgagcgcaagttgaatctgcaaatacgaggagtcctgttgatgttgccagtaaaattagcaggtgttctagtcctgtggccatctgcctagtaaagctttttgcatgaaccttctatgaatgttactgttttatttttagaaatgtcagttgctgcgtccccagacttttgatttgcactatgagcctataggccagcctaccctctggtagattgtcgcttatcttgtaagaaaaacaaatctcttaaattaccacttttaaataataatactgagattgtatctgtaagaaggatttaaagagaagctatattagttttttaattggtattttaatttttatatattcaggagagaaagatgtgattgatattgttaatttagacgagtctgaagctctcgatttcctatcagtaacagcatctaagaggttttgctcagtggaataaacatgtttcagcagtgttggctgtattttcccactttcagtaaatcgttgtcaacagttccttttaaatgcaaataaataaattctaaaaatt

SEQ ID NO: 10 - protein, 375 aa, Mus musculus
MPTRSPSVVISDDEPGYDLDLFCIPNHYAEDLEKVFIPHGLIMDRTERLARRWHQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQVPGSFVSRDVMKEMGGHHIVALCVLKGGYKFFADLLDYIKALNRNSDRSIPMTVDFIRLKSYCNDQSTGDIKVIGGDDLSTLTGKNVLIVEDIIDTGKTMQTLLSLVKQYSPKMVKVASLLVKRTSRSVGYRPDFVGFEIPDKFVVGYALDYNEYFRNLNHVCVISETGKAKYKA